(19) United States
(12) Patent Application Publication
(10) Pub. No.: US 2016/0355781 A1
(43) Pub. Date: Dec. 8, 2016

(54) SYSTEMS, METHODS, AND CELLULAR COMPOSITIONS THEREOF, INVOLVING INTRODUCTION, ATTACHMENT AND PROLIFERATION OF TROPHECTODERM CELLS IN A BLASTOCYST

(71) Applicant: Texas Health Biomedical Advancement Center, Inc., Arlington, TX (US)

(72) Inventor: Oscar Perez, Frisco, TX (US)

(73) Assignee: Texas Health Biomedical Advancement Center, Inc., Arlington, TX (US)

(21) Appl. No.: 15/173,521

(22) Filed: Jun. 3, 2016

Related U.S. Application Data
(60) Provisional application No. 62/170,504, filed on Jun. 3, 2015.

(51) Int. Cl. C12N 5/073; A61B 17/43; C12M 1/34; C12N 13/00; C12M 3/00

(52) U.S. Cl. CPC C12N 5/0603; C12N 13/00; C12M 21/06; C12M 41/46; A61B 17/43; C12N 2502/025

(57) ABSTRACT

A method of improving quality of a blastocyst is described. The method includes removing a segment of trophectoderm from a first blastocyst. The method also includes removing a portion of trophectoderm from a second blastocyst. In addition, the method includes introducing to the second blastocyst the segment of trophectoderm from the first blastocyst. The method will promote attachment and proliferation of the trophectoderm in the second blastocyst. The first and second blastocyst will often be obtained from a same or similar period of time after insemination. The first blastocyst will have a same quality or better quality trophectoderm than the second blastocyst. The second blastocyst will include the segment of trophectoderm from the first blastocyst.

### CROSS REFERENCE TO RELATED APPLICATIONS

This application claims priority to U.S. Provisional Patent Application No. 62/170,504 filed on Jun. 3, 2015, all of which is hereby incorporated by reference for all purposes, and to the maximal extent allowable.

### FIELD OF THE INVENTION

The invention describes novel systems, methods, and compositions thereof, which provide improved blastocysts for transfer, and provide improved blastocysts with a higher outcome of success with transfer.

### BACKGROUND

In very early development of mammals, there is formation of a blastocyst, a structure comprising an inner cell mass (ICM), an inner blastocyst cavity, and outer trophoblasts or trophectoderm cells. While the exact day of development of the blastocyst varies in certain mammals, the sequence of events in embryonic development is predictable, and it is understood that after shedding (hatching), the blastocyst becomes implantation competent (e.g., capable of becoming physically and physiologically intimate with the uterus). Regardless of the type of implantation, its purpose is the same, which is to provide apposition, and attachment, with the uterus, and eventually to allow for a blood supply between maternal blood vessels and developing blood vessels.

Currently, assisted reproductive technology (ART), which includes in vitro fertilization (IVF) and variations thereof (e.g., gamete intrafallopian transfer or GIFT), is considered an inefficient option to obtain a clinical pregnancy and birth. To be successful, eggs must be retrieved and then fertilized, and then continue to divide and grow for a number of days ex vivo before being suitable for transfer (e.g., transfer to a uterus). In many present procedures, it is when the fertilized egg forms the blastocyst (e.g., about day 5, or about day 6 after fertilization/insemination) that transfer is initiated. In other presently used procedures, it is a few days after the fertilized egg forms the blastocyst that transfer is initiated. And, in still other procedures, transfer is initiated during the early cleavage or a pre-blastocyst stage (cleavage stage transfer), which is at about day 2 or day 3 after fertilization/insemination, in which there are generally less than 32 cells. In all such cases, however, transfer is often unsuccessful, and is reduced with age.

Successful blastocyst transfer requires several things, including a good quality blastocyst, a good culture system, and good laboratory experience. Unfortunately, such conditions are not always met, which is further complicated by variations in patient population and patient conditions. For example, retrieval of a good quality blastocyst is lower in an older female (e.g., 30s and older), as fewer eggs and/or good quality eggs are obtained, and there are fewer blastocysts selectable and/or viable for transfer. In addition, there are other factors, such as maturity of the egg, fertilization/insemination itself, and ex vivo conditions, to name a few factors, that also play a large in role in the poor success rate of ART. In fact, presently, many, if not most, blastocysts that form after retrieval are being discarded. Using humans as an example, approximately 80% of blastocysts retrieved from humans are being discarded because they do not reach an optimal quality that is believed to provide a good chance of a clinical pregnancy or a birth outcome. Presently, blastocyst quality is often expressed in terms of overall quality, which includes the number and quality of trophectoderm cells as well as the ICM and the quality of the inner blastocyst cavity, and each such blastocyst at this stage of development is given a grade. Transfer of only certain grades of blastocysts have been associated with higher implantation and pregnancy rates. For example, expanding and/or hatching blastocysts, which are of a certain grade, have been associated with higher implantation and pregnancy rates. On the other hand transfer of early blastocysts or poor quality blastocysts, and which are of a different grade, have been associated with and found to yield low to very low implantation and pregnancy rates. Because many or most blastocysts are graded low (e.g., mediocre, fair, or poor) rather than graded high (e.g., good), most blastocysts are being discarded. As a result, there remains a need to improve blastocyst quality and/or outcome, as well as a need to provide culture conditions that provide a more successful outcome for such improved blastocysts.

### OVERVIEW

In one or more embodiments described herein are systems and methods that provide improved blastocysts for transfer. The systems and methods described herein provide improved blastocysts with a higher outcome of success with transfer. The systems and methods described herein include an introduction, attachment and proliferation of trophectoderm cells in blastocysts, all or a portion of which may occur ex vivo.

In one or more embodiments described herein are systems and methods for attaching and proliferating trophectoderm cells in a blastocyst. The systems and methods for attaching and proliferating trophectoderm cells in a blastocyst provide an improved blastocyst for transfer. The systems and methods for attaching and proliferating trophectoderm cells in a blastocyst provide an improved blastocyst with a higher outcome of success with transfer.

Embodiments disclosed herein provide a method and/or a means for introducing new trophectoderm cells in a blastocyst. Embodiments disclosed herein provide a method and/or a means for successful attachment of newly introduced trophectoderm cells in the blastocyst. Embodiments disclosed herein provide a method and/or a means for inducing proliferation of trophectoderm cells in the blastocyst.

In one or more embodiments, the blastocyst as described herein (e.g., the blastocyst to which new trophectoderm cells are introduced) includes a blastocyst that may, for whatever reason, contain a low number of trophectoderm cells, or lower than usual number of trophectoderm cells. In addition, or as an alternative, the blastocyst may be a blastocyst having, for example, at least one of the following: (i) a poor trophectoderm score (e.g., few or very few cells forming a cohesive epithelium) in accordance with a multivariate analysis model of trophectoderm morphology and/or a morphological grading system of the trophectoderm and/or the blastocyst; (ii) a fair or mediocre trophectoderm score (e.g., few cells forming a cohesive epithelium) in accordance with a multivariate analysis model of trophectoderm morphology and/or a morphological grading system of the trophectoderm and/or the blastocyst; (iii) a Grade B in trophectoderm quality (e.g., several cells organized in a loose epithelium) in accordance with a standardized approach for grading a blastocyst and/or the trophectoderm of a blastocyst (e.g., Gardner scale, and/or SART approach); (iv) a Grade C in trophectoderm quality (e.g., few large cells) in accordance with a standardized approach for grading a blastocyst and/or the trophectoderm of a blastocyst (e.g., Gardner scale, and/or SART approach); (v) a Grade D in trophectoderm quality (e.g., few cells and/or degenerative cells) in accordance with a standardized approach for grading a blastocyst and/or the trophectoderm of a blastocyst (e.g., Gardner scale, and/or SART approach).

In one or more embodiments, trophectoderm cells are removed from a donor blastocyst having donor cells, and are introduced into a receiver blastocyst having receiver cells, in which the donor blastocyst has, as compared with the receiver blastocyst, at least one of the following: (i) a same or higher blastocyst rating; and/or (ii) a same or higher trophectoderm score in accordance with a multivariate analysis model of trophectoderm morphology and/or a morphological grading system of the trophectoderm; and/or (iii) a same or better trophectoderm quality in accordance with a grading system and/or a standardized approach for grading the trophectoderm of the blastocyst; and/or (iv) a same or better grade for the trophectoderm in accordance with or based on a grading system and/or a standardized approach for grading the trophectoderm of the blastocyst; and/or (v) a same or better grade for the blastocyst in accordance with or based on a grading system and/or a standardized approach for grading the blastocyst.

In one or more embodiments, the trophectoderm cells (e.g., donor cells) removed from the donor blastocyst do not include the ICM.

In one or more embodiments, the donor blastocyst and the receiver blastocyst are from a same fertilization/insemination (e.g., from one parental pair), and do not provide a germ line genetic modification (e.g., genetic hybrid with more than one parental pair).

The receiver blastocyst may, as compared with the donor blastocyst, have at least one of the following: (i) a low (or lower) number of trophectoderm cells; and/or (ii) a low (or lower) quality of the trophectoderm; and/or (iii) a low (or lower) blastocyst rating; and/or (iv) a low (or lower) trophectoderm score; and/or (v) a low (or lower) grade of the trophectoderm; (vi) and/or a low (or lower) grade of the blastocyst. Introducing the donor cells from the donor blastocyst using the systems and methods disclosed herein have proven to provide a catalyzing reaction in the receiver blastocyst, whereby the receiver blastocyst is initiated into generating, and subsequently generates, its own trophectoderm cells. In one or more embodiments, the catalyzing reaction initiates and/or includes attachment of newly introduced trophectoderm cells (e.g., donor cells) in the receiver blastocyst (e.g., the blastocyst having the low number, or lower number of trophectoderm cells). In one or more embodiments, the catalyzing reaction includes proliferation of trophectoderm cells in the receiver blastocyst. In one or more embodiments, the catalyzing reaction may provide an increase in viability of the receiver blastocyst. In one or more embodiments, the catalyzing reaction, and/or introducing the newly introduced trophectoderm cells (e.g., donor cells) to the receiver blastocyst provides to the receiver blastocyst at least one of the following (as or when compared with the receiver blastocyst before the catalyzing reaction, and/or before introducing the donor cells to the receiver blastocyst): (i) a high (or higher) number of trophectoderm cells; and/or (ii) a high (or higher) quality of the trophectoderm; (iii) a high (or higher) trophectoderm grade/score; (iv) a high (or higher) ICM grade/rating; and/or (iv) a high (or higher) blastocyst grade/rating.

In one or more embodiments, the systems and methods described herein, including the catalyzing reaction thereof, provide a rescue mechanism for a blastocyst having a low (or lower) blastocyst rating, and/or a low (or lower) trophectoderm morphology, and/or a low (or lower) trophectoderm quality, and/or a low (or lower) trophectoderm grade. In one or more embodiments, the systems and methods described herein, including the catalyzing reaction thereof, provide a cellular composition having undergone the catalyzing reaction. In one or more embodiments, the systems and methods described herein, including the catalyzing reaction thereof, provide a cellular composition having undergone the catalyzing reaction, the cellular composition being in the form of a catalyzed blastocyst. In one or more embodiments, the systems and methods described herein, including the catalyzing reaction thereof, provide a cellular composition having undergone the catalyzing reaction, the cellular composition being in the form of a rescued blastocyst, the rescued blastocyst being a viable blastocyst. In one or more embodiments, the systems and methods described herein, including the catalyzing reaction thereof, provide a cellular composition as a viable embryo. In one or more embodiments, the systems and methods described herein, including the catalyzing reaction thereof, provide a cellular composition as a rescued blastocyst for transfer, offering to said blastocyst, as a viable embryo, an opportunity for transfer and/or for successful clinical pregnancy and/or birth. In one or more embodiments, the systems and methods described herein, including the catalyzing reaction thereof, provide a cellular composition or rescued blastocyst, or suitable embryo that is viable for a transfer to a uterus. The transfer may be a single blastocyst transfer, in which the single blastocyst is the cellular composition described herein (e.g., a rescued or catalyzed blastocyst). The transfer may be a double blastocyst transfer, in which at least one of the blastocysts is a cellular composition as described herein (e.g., a rescued or catalyzed blastocyst). The transfer may be a double blastocyst transfer, in which both of the blastocysts are cellular compositions as described herein (e.g., a rescued or catalyzed blastocyst). In one or more embodiments, the systems and methods described herein, including the catalyzing reaction thereof, provide a cellular composition or rescued blastocyst or suitable embryo as described herein that is viable for a fresh transfer to a uterus. In one or more embodiments, the systems and methods described herein, including the catalyzing reaction thereof, provide a cellular composition or rescued blastocyst or suitable embryo as described herein that is viable for transfer after cryopreservation.

Embodiments disclosed herein further provide a method and/or a means for providing and/or determining viability of a cellular composition as described herein (e.g., a rescued blastocyst). In one or more embodiments, the method and/or a means for providing and/or determining viability of the cellular composition described herein includes a period of incubation of the receiver blastocyst after introducing the donor cells to the receiver blastocyst. The period of incubation may be from about 14 hours to about 28 hours, or any period of time therebetween or thereabout. In one embodiment, the period of incubation is about 22 hours. In another embodiment, the period of time is within or up to about 24 hours. In other embodiments, the period of incubation is within or up to about 24 hours after attachment of the donor cells to the receiver blastocyst. The average trophectoderm cells require approximately 14 to 26 hours to proliferate. In one or more embodiments, the method and/or a means for providing and/or determining viability of the cellular composition described herein includes a period of proliferation of trophectoderm cells of the receiver blastocyst after introducing the donor cells to the receiver blastocyst. In one or more embodiments, the period of proliferation is about or is up to about 24 hours after attachment of the donor cells to the receiver blastocyst.

In one or more embodiments is a method of improving quality of a blastocyst and/or of rescuing a blastocyst, the method comprising removing a segment of trophectoderm from a first blastocyst. The method further comprises removing a portion of trophectoderm from a second blastocyst. The method further comprises introducing to the second blastocyst the segment of trophectoderm from the first blastocyst. In one or more embodiments, the segment of trophectoderm from a first blastocyst does not include cells from an inner cell mass of the first blastocyst. The first blastocyst will have a same quality or better quality trophectoderm than the second blastocyst. The first blastocyst may have a better quality trophectoderm that the second blastocyst. The segment of trophectoderm may comprise a plurality of trophectoderm cells, at least some of which are united by tight junctions. The segment of trophectoderm may comprise a plurality of trophectoderm cells united by tight junctions. The segment of trophectoderm may comprise a plurality of trophectoderm cells, the plurality of trophectoderm cells ranging from about two cells to about twelve cells, or any range therebetween or thereabout. The method may further comprise breaking a surface of the first blastocyst to remove the segment of trophectoderm from the first blastocyst. The method may further comprise initially extending the segment of trophectoderm away from a zona pellucida of the first blastocyst before removing the segment of trophectoderm from the first blastocyst. The removing the segment of trophectoderm from the first blastocyst may comprise aligning light emitted from a laser on a first region of the segment of trophectoderm of the first blastocyst. The removing the segment of trophectoderm from the first blastocyst may comprise pulsing light at a frequency and wavelength sufficient to break a portion of the first region. The removing the segment of trophectoderm from the first blastocyst may comprise aligning light emitted from the laser on a second region of the segment of trophectoderm of the first blastocyst. The removing the segment of trophectoderm from the first blastocyst may comprise pulsing light at the same or similar frequency and wavelength sufficient to break a portion of the second region. The aligning light may differ from the pulsing light. The removing the portion of trophectoderm from the second blastocyst may comprise aligning light emitted from a laser on a first region of the portion of trophectoderm of the second blastocyst. The removing the portion of trophectoderm from the second blastocyst may comprise pulsing light at a frequency and wavelength sufficient to break a portion of the first region. The removing the portion of trophectoderm from the second blastocyst may comprise aligning light emitted from the laser on a second region of the portion of trophectoderm of the second blastocyst. The removing the portion of trophectoderm from the second blastocyst may comprise pulsing light at the same or similar frequency and wavelength sufficient to break a portion of the second region. The first blastocyst may have a grade of B or C, the grade in accordance with a Gardner grading system or adapted from a Gardner grading system. The second blastocyst may have a grade of C or D, the grade in accordance with a Gardner grading system or adapted from a Gardner grading system. The laser may include a high power laser diode emitting light at a wavelength of about 1400 nm, or from about 1000 nm to 2000 nm. The laser may include a red laser diode emitting light at a wavelength of about 600-650 nm. The portion of trophectoderm from the second blastocyst may include at or about 3 trophectoderm cells to about 7 trophectoderm cells, or from about 4 to 5 trophectoderm cells. The method may further comprise placing the first blastocyst and the second blastocyst in a blastocyst media, the blastocyst media further comprising an addition of protein in an amount that is from about 10% to about 30%, or is about 20%. The may further comprise placing the second blastocyst in a blastocyst media after introducing to the second blastocyst the segment of trophectoderm from the first blastocyst, the blastocyst media further comprising an addition of protein in an amount that is from about 10% to about 30%, or is about 20%. The method may further comprise incubating the second blastocyst for about 14 hours to about 26 hours after the segment of trophectoderm was introduced to the second blastocyst. The method may further comprise contacting trophectoderm of the second blastocyst with at least a portion of the segment of trophectoderm when introducing the segment of trophectoderm to the second blastocyst. The removing a portion of trophectoderm from the second blastocyst provides access into the second blastocyst, and the access may be enlarged by a high power laser ablation.

In one or more embodiments is a blastocyst having an improved quality and/or having been rescued, the blastocyst being a first blastocyst comprising an absence of a portion of a zona pellucida, and an absence of a portion of a trophectoderm, the absence of the portion of the zona pellucida and the absence of the portion of the trophectoderm forming a gap. The first blastocyst may further comprise replacement trophectoderm from a second blastocyst, the replacement trophectoderm comprising trophectoderm of a same or better quality, the replacement trophectoderm located at or adjacent the gap. The second blastocyst may be obtained from a same or similar period of time after insemination as the first blastocyst. The gap may comprise trophectoderm cells from the second blastocyst, the trophectoderm cells from the second blastocyst being of a better quality or having a higher number than trophectoderm cells from the portion of the trophectoderm now absent in the first blastocyst. The gap may be formed by a laser or a laser ablation technique as described herein.

In one or more embodiments is a blastocyst system for improving quality of at least one blastocyst and/or for rescuing at least one blastocyst provided initially as a pair of blastocysts, the system and/or pair of blastocysts comprising a first blastocyst have a first quality trophectoderm, at least a segment of the first quality trophectoderm being accessible and removable for transfer to a second blastocyst. The blastocyst system may further comprise the second blastocyst having a second quality trophectoderm, the second quality trophectoderm being a same quality or less quality than the first quality trophectoderm of the first blastocyst, a portion of the second quality trophectoderm removable from the second blastocyst and replaced by the segment of the first quality trophectoderm of the first blastocyst. The first blastocyst and the second blastocyst may be from a same or similar period of time after insemination. The segment may be removed from the first blastocyst by a laser or a laser ablation technique as described herein. The portion of the second quality trophectoderm may be removed from the second blastocyst by a laser or a laser ablation technique as described herein. The blastocyst system may further comprise a microscopic system for at least visualizing the first and second blastocysts, the microscopic system comprising micromanipulation tools cooperative with a microinjection system. The blastocyst system may further comprise a laser system for at least removing the segment of the first quality trophectoderm from the first blastocyst, and for removing the portion of the second quality trophectoderm from the second blastocyst. The blastocyst system may further comprise a computing system for at least obtaining and storing data about the first and second blastocysts. The blastocyst system may further comprise a culturing system for at least removing the segment of the first quality trophectoderm from the first blastocyst, and for removing the portion of the second quality trophectoderm from the second blastocyst.

In one or more embodiments is a system for improving quality of at least one blastocyst and/or for rescuing at least one blastocyst provided initially as a pair of blastocysts, the system and/or pair of blastocysts comprising a first blastocyst having first quality trophectoderm cells, and a second blastocyst having second quality trophectoderm cells. The system may further comprise a microscopic system for visualizing the first blastocyst and the second blastocyst. The system may further comprise a micromanipulation system cooperative with at least the microscopic system, the micromanipulation system further comprising a microinjection system, the micromanipulation system and the microinjection system for manipulating the first quality trophectoderm cells of the first blastocyst, and the second quality trophectoderm cells of the second blastocyst, wherein the first quality trophectoderm cells are better quality or grade than the second quality trophectoderm cells. The system may further comprise a laser system for removing a segment comprising at least some of the first quality trophectoderm cells from the first blastocyst, and for removing a portion of the second quality trophectoderm cells from the second blastocyst, the laser system cooperative with at least the microscopic system. The system may further comprise a culturing system for maintaining the first blastocyst and the second blastocyst during manipulation with the micromanipulation system, and for incubating the second blastocyst after the manipulation. The system may further comprise a computing system cooperative with at least the microscopic system for at least obtaining and storing data about the first and second blastocysts visualized by the microscopic system.

In one or more embodiments is a receiver blastocyst containing trophectoderm cells obtained from a donor blastocyst, the donor blastocyst and the receiver blastocyst from a same fertilization, the donor blastocyst having one or more of a better quality trophectoderm or more trophectoderm cells than the receiver blastocyst before the trophectoderm cells were obtained from the donor blastocyst.

In one or more embodiments is a blastocyst having an improved quality and/or having been rescued, the blastocyst being a first blastocyst comprising an absence of a portion of a zona pellucida, and an absence of a portion of a trophectoderm, the absence of the portion of the zona pellucida and the absence of the portion of the trophectoderm forming a gap. The blastocyst further comprises replacement trophectoderm from a second blastocyst located at or adjacent the gap, the replacement trophectoderm comprising trophectoderm of a same or better quality. The first and second blastocysts are obtained from one or more of a same or similar period of time after fertilization, and a same fertilization. The replacement trophectoderm may include up to ten or up to twelve trophectoderm cells and zona pellucida from the second blastocyst, wherein, in addition to the replacement trophectoderm, the second blastocyst has a higher number of trophectoderm cells than the first blastocyst.

In one or more embodiments is a blastocyst system for improving quality of at least one blastocyst and/or for rescuing at least one blastocyst provided initially as a pair of blastocysts, the system and/or pair of blastocysts comprising a first blastocyst having first quality trophectoderm cells, and a second blastocyst having second quality trophectoderm cells. The first blastocyst having a first quality trophectoderm has at least a segment of the first quality trophectoderm removed from the first blastocyst. In one or more embodiments, the segment does not contain cells from an inner cell mass of the first blastocyst. The second quality trophectoderm has one or more of fewer trophectoderm cells than the first blastocyst, a same quality trophectoderm as the first blastocyst, and a lesser quality trophectoderm than the first blastocyst. The second blastocyst further comprises the segment from the first blastocyst introduced in the second quality trophectoderm. The first and second blastocysts are obtained from one or more of a same or similar period of time after fertilization, and a same fertilization. The blastocyst system may further comprise a microscopic system for at least visualizing the first and second blastocysts, the microscopic system comprising micromanipulation tools cooperative with a microinjection system. The blastocyst system may further comprise a laser system for at least removing the segment of the first quality trophectoderm from the first blastocyst, and for ablating a portion of the zona pellucida of the second blastocyst. The blastocyst system may further comprise a computing system for at least obtaining and storing data about the first and second blastocysts. The blastocyst system may further comprise a culturing system for at least culturing the first and second blastocysts, the culturing system including a blastocyst media further comprising an addition of protein in an amount that is from about 10% to about 30%, or is about 20%.

These and other embodiments are described further below.

### BRIEF DESCRIPTION OF DRAWINGS

Embodiments are illustrated by way of example in the accompanying figures not necessarily drawn to scale, in which like numbers indicate similar parts, and in which:

FIG. 1A depicts a representative blastocyst as described herein.

FIG. 1B illustrates a representative grading system as described herein.

FIG. 2 illustrates a representative system described herein, which includes a representative microscopic system as described herein.

FIG. 3 illustrates representative components and/or units of a representative micromanipulator system described herein, which is suitable with and may be associated with a system described herein, which includes the representative microscopic system depicted in FIG. 2.

FIGS. 4A, 4B, 4C and 4D illustrates representative components and/or units of a representative laser system described herein, which is suitable with and may be associated with a system, which includes a representative microscopic system, such as depicted in FIG. 2, and a representative micromanipulator system, such as depicted in FIG. 3.

FIGS. 5A and 5B illustrate representative micromanipulator needles and components associated with a micromanipulator system described herein, such as the representative micromanipulator system depicted in FIG. 3.

FIGS. 6A, 6B, and 6C illustrate further details regarding representative micromanipulator needles and components described herein.

FIG. 7A illustrates another view of a representative system described herein, which includes the representative microscopic system depicted in FIG. 2.

FIG. 7B is an expanded view of software associated with the system depicted in FIG. 7A.

FIG. 8 shows a representative component of a culturing system described herein, and suitable with a system described herein.

FIGS. 9A and 9B show still further representative components of the culturing system described herein.

FIG. 10 illustrates an overall flow chart as described herein.

FIG. 11 depicts a further and representative flow chart as described herein.

FIG. 12 depicts another representative flow chart as described herein.

FIG. 13 depicts still another representative flow chart as described herein.

FIG. 14, comprising FIGS. 14A-14K, illustrate representative steps of methods described herein.

FIGS. 15A and 15B illustrate representative blastocysts selected as described herein, and laser positioning on said blastocysts as described herein.

FIG. 15C is a representative laser utilized with FIGS. 15A and 15B.

### DETAILED DESCRIPTION

Although making and using various embodiments are discussed in detail below, it should be appreciated that as described herein are provided many inventive concepts that may be embodied in a wide variety of contexts. Embodiments discussed herein are merely representative and do not limit the scope of the invention.

It is hereby proposed that trophectoderm cells may be a better predictor of success of a blastocyst than cells of the inner cell mass (ICM). Accordingly, as described herein, the quality of the trophectoderm in a blastocyst, and/or the number and/or quality of trophectoderm cells in a blastocyst, is improved by manipulating the trophectoderm and the trophectoderm cells of that blastocyst.

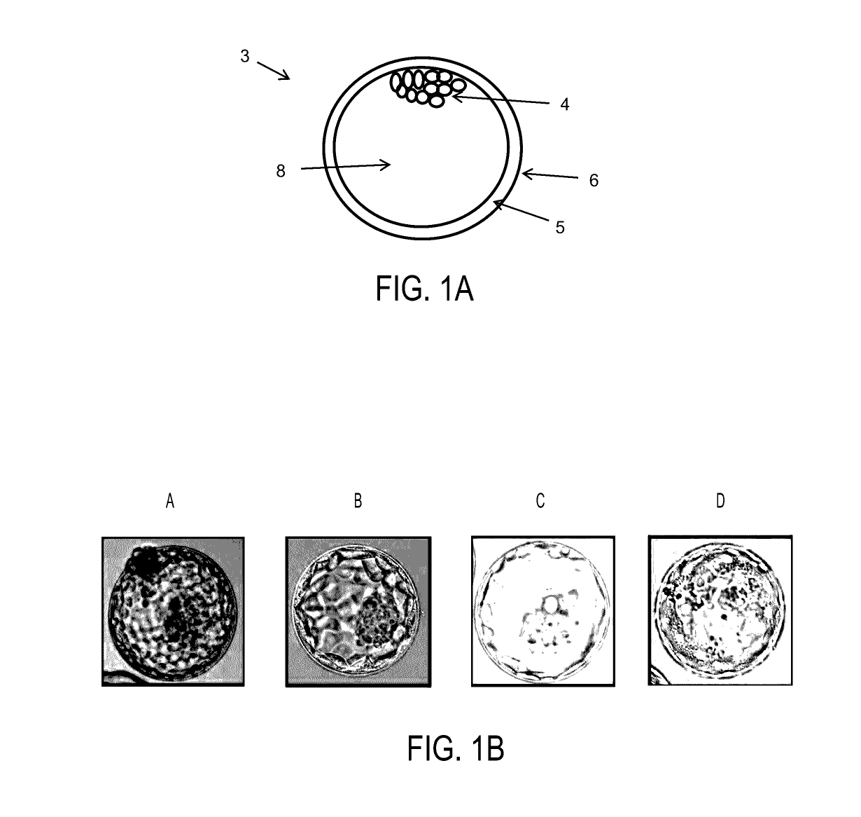

Any blastocyst as described herein is a blastocyst that has undergone polarization, and loss of totipotency, and, as represented in FIG. 1A, is a blastocyst 3 having inner mass cells 4 and a trophectoderm 6 encased by a zona pellucida 5. In one or more embodiments, such a blastocyst 3 has two cell types: (a) the inner mass cells 4; and (b) the trophectoderm cells that are in the trophectoderm 6 (generally as a single layer of cells), in which the trophectoderm cells are forming and/or have formed tight junctions between said trophectoderm cells, and which together (the trophectoderm cells with or without tight junctions) form a surrounding within which is an inner or cystic (fluid-filled) cavity 8 and the inner mass cells 4.

Generally, any blastocyst as described and utilized herein will not include a pre-blastocyst, or one that is developmentally identified as being in a morula stage (e.g., having eight cells or less than eight cells).

Any of the blastocysts described and utilized herein may be derived from an in vitro fertilization process. Any of the blastocysts described and utilized herein may be derived from an artificial insemination process.

Generally, any blastocyst described and utilized herein will be initially at a period post-fertilization/insemination that is in a range from between about three days post-fertilization/insemination and about seven days post-fertilization/insemination. In one or more embodiments, any blastocyst described herein will initially have a number of cells that is from about fifty cells to about 200 cells, or in any range therebetween or thereabout. In one or more embodiments, any blastocyst described herein will initially have a number of cells that is from about sixty cells to 300 cells or less, or in any range therebetween or thereabout.

In one or more embodiments, any blastocyst described herein will initially be at a period that is about three days post-fertilization/insemination. In one or more embodiments, any blastocyst described herein will initially be at a period that is about four days post-fertilization/insemination. In one or more embodiments, any blastocyst described herein will initially be at a period that is about five days post-fertilization/insemination. In one or more embodiments, any blastocyst described herein will initially be at a period that is about six days post-fertilization/insemination. In one or more embodiments, any blastocyst described herein will initially be at a period that is about seven days post-fertilization/insemination.

In one or more embodiments, any blastocyst described herein may be from unused fertilized eggs obtained via in-vitro fertilization and/or artificial insemination, in which there is consent for use and manipulation of the unused fertilized eggs.

One blastocyst being manipulated, as described herein, is a receiver blastocyst.

One blastocyst being manipulated, as described herein, is a donor blastocyst.

In one or more embodiments, a receiver blastocyst as described herein is a blastocyst that has been identified as an expanding, expanded, and/or hatching blastocyst with a low number of trophectoderm cells, as is it understood in the field, and/or as is described below, including any one or more of TABLES 1A, 1B, 1C, 2A, 2B, 2C, 3A, 3B, 3C, and 4. In one or more embodiments, a receiver blastocyst as described herein is a blastocyst that has been identified as an expanding, expanded, and/or hatching blastocyst (as is it understood in the field, and/or as is described below, including any one or more of TABLES 1A, 1B, 1C, 2A, 2B, 2C, 3A, 3B, 3C, and 4), and has a lower number of trophectoderm cells and/or lower quality trophectoderm as compared with a donor blastocyst.

In one or more embodiments, a donor blastocyst as described herein is a blastocyst that has been identified as an expanding, expanded, and/or hatching blastocyst with a high number of trophectoderm cells, as is it understood in the field, and/or as is described below, including any one or more of TABLES 1A, 1B, 1C, 2A, 2B, 2C, 3A, 3B, 3C, and 4. In one or more embodiments, a donor blastocyst as described herein is a blastocyst that has been identified as an expanding, expanded, and/or hatching blastocyst (as is it understood in the field, and/or as is described below, including any one or more of TABLES 1A, 1B, 1C, 2A, 2B, 2C, 3A, 3B, 3C, and 4), and has a higher number of trophectoderm cells and/or higher quality trophectoderm as compared with a receiver blastocyst.

In one or more embodiments, the receiver blastocyst as described herein is a blastocyst, as described above (and/or as is described below, including any one or more of TABLES 1A, 1B, 1C, 2A, 2B, 2C, 3A, 3B, 3C, and 4), having been fertilized (and/or inseminated) in vivo or ex vivo, and having one or more features that identify the receiver blastocyst as being any one of: (i) unsuitable for transfer; (ii) less suitable for transfer; (iii) non-viable for transfer; and/or (iv) less viable for transfer; in which transfer utilizes one or more of a transfer technique for a blastocyst and/or a portion of a fertilization/transplantation technique for a blastocyst; and/or (v) discarded. Any individual or combination of (i), (ii), (iii), (iv), and/or (v) may be utilized to identify a receiver blastocyst for manipulation as described herein. In one or more embodiments, the receiver blastocyst selected for manipulation as described herein is (or has been, or will be) discarded, and, thereby, is not or will not be considered suitable for transfer, and/or for a fertilization/transplantation technique, and/or will not be utilized for transfer, and/or for a fertilization/transplantation technique.

In one or more embodiments, the receiver blastocyst as described herein is a blastocyst that has been or will be discarded, as is it understood in the field, and/or as is described below. In one or more embodiments, the receiver blastocyst as described herein is a blastocyst that has been or will be identified as a discarded blastocyst and has a low number of trophectoderm cells, and/or as is unsuitable for transfer as described below, and/or as exemplified in any one or more of TABLES 1A, 1B, 1C, 2A, 2B, 2C, 3A, 3B, 3C, and 4.

In one or more embodiments, the donor blastocyst as described herein is a blastocyst that has been or will be discarded, as is it understood in the field, and/or as is described below. In one or more embodiments, the donor blastocyst as described herein is a blastocyst that has been or will be suitable for transfer as described below, and/or as exemplified in any one or more of TABLES 1A, 1B, 1C, 2A, 2B, 2C, 3A, 3B, 3C, and 4. In one or more embodiments, the donor blastocyst as described herein is a blastocyst that has been or will be identified as: (i) suitable for transfer, and has a good number of trophectoderm cells; (ii) suitable for transfer, and has a good quality trophectoderm; (iii) viable for transfer, and has a good number of trophectoderm cells; (iv) viable for transfer, and has a good quality trophectoderm; (v) has a good number of trophectoderm cells; (vi) has a good quality trophectoderm; (vii) has a number of trophectoderm cells that is the same or greater than the number of trophectoderm cells of the receiver cell; and/or (viii) has a trophectoderm quality that is the same or greater than the quality of the trophectoderm of the receiver cell. Any individual or combination of (i), (ii), (iii), (iv), (v), (vi), (vii), and/or (viii) may be utilized to identify a donor blastocyst for manipulation as described herein.

In one or more embodiments, the donor blastocyst selected for manipulation as described herein is (or has been, or will be) discarded, and, thereby, is not or will not be considered suitable for transfer, and/or for a fertilization/transplantation technique, and/or will not be utilized for transfer, and/or for a fertilization/transplantation technique; however, this donor blastocyst that is or has been or will be discarded will have any one of: (a) a same, substantially the same, or higher number of trophectoderm cells than the receiver blastocyst; and/or (b) a same, substantially the same, or higher quality trophectoderm than the receiver blastocyst.

Suitability of a blastocyst (e.g., for transfer, and/or for a fertilization/transplantation technique) is generally based on a grading or scoring system, such as is known in the field. In one or more embodiments, the grading or scoring system may include at least a quality of the trophectoderm of the blastocyst. In addition, or as an alternative, the grading system may include at least a number and/or quality of trophectoderm cells of the blastocyst. In addition, or as an alternative, the grading system may include at least a quality of the blastocyst. In addition, or as an alternative, the grading system may include at least a quality of the blastocyst and the number of cells and/or quality of the trophectoderm of the blastocyst.

Such grading or scoring systems often include an expansion grading system of the blastocyst as represented in any of TABLES 1A and 1B and 1C, and/or of the inner cell mass (ICM) as represented in any of TABLES 2A and 2B and 2C, and/or of the trophectoderm as represented in any of TABLES 3A and 3B and 3C, or any combination thereof. The present grading or scoring system are based or adapted from a Gardner grading system, which is known in the field, an example of which is in at least the following: Gardner D, et al., Fertility and Sterility, 2000, vol. 73(6), pp. 1155-1158; and Gardner D, et al., In vitro culture of human blastocysts. In: Toward reproductive certainty (fertility and genetics beyond 1999) (Carnforth, Parthenon Publishing), 1999, pp. 378-88, each of which is incorporated herein by reference in its entirety. For example, a first representative grading system adapted from the Gardner grading system may include TABLES 1A (for the blastocyst), 2A (for the ICM), and 3A (for the trophectoderm). A second grading system that is closely in line with the Gardner grading system may include TABLES 1B (for the blastocyst), 2B (for the ICM), and 3B (for the trophectoderm). A third grading system that is adapted from the Gardner grading system may include TABLES 1C (for the blastocyst), 2C (for the ICM), and 3C (for the trophectoderm).

TABLE 1A

| Grade | Description (Blastocyst) |
|---|---|
| 1 | Early Blastocyst: the fluid-filled cavity (e.g., blastocoel) filling |
| | more than ½ the total volume (but generally no expansion in |
| | overall size compared to earlier stages) |
| 2 | Blastocyst: the fluid-filled cavity (e.g., blastocoel) filling more |
| | than ½ the total volume, with slight expansion in overall size, |
| | generally accompanied by a notable thinning of the zona |
| 3 | Full Blastocyst: the fluid-filled cavity (e.g., blastocoel) filling |
| | more than 50% of the total volume and overall size fully |
| | enlarged with a very thin zona |
| 4 | Hatching Blastocyst: trophectoderm has started to herniate |
| | through the zona |
| 5 | Fully Hatched Blastocyst: free blastocyst fully removed from the |
| | zona |

TABLE 2A

| Grade | Description (ICM) |
|---|---|
| A | Tightly packed compacted cells |
| B | Large, loose cells |
| C | No ICM distinguishable |
| D | Cells of the ICM appear degenerative |

TABLE 3A

| Grade | Description (Trophectoderm) |
|---|---|
| A | Many healthy cells forming a cohesive epithelium |
| B | Few but healthy cells, large in size |
| C | Poor, unevenly distributed cells; many appear as few cells |
| | squeezed to the side |
| D | Cells of the trophectoderm appear degenerative on the surface |

TABLE 1B

| Grade | Description (Blastocyst) |
|---|---|
| 1 | The fluid-filled cavity takes up less than half the space or less |
| | than half the total volume (defined by the zona) |
| 2 | The fluid-filled cavity takes up more than half the space or less |
| | than half the total volume (defined by the zona) |
| 3 | The blastocyst cavity has expanded into the entire volume |
| | defined by the zona, pressing the trophectoderm cells up |
| | tightly against the inside of the zona. |
| 4 | The expanded blastocyst, where the blastocyst has increased |
| | beyond the original volume defined by the zona, and caused |
| | the zona pellucida “shell” to become super thin |
| 5 | The zona is breached, and blastocyst is hatching out of the |
| | envelope |
| 6 | The blastocyst is completely hatched, and is free of the zona |
| | (zona is empty) |

TABLE 2B

| Grade | Description (ICM) |
|---|---|
| A | Many cells, tightly packed |
| B | Several cells, loosely grouped |
| C | Very few or no cells |

TABLE 3B

| Grade | Description (Trophectoderm) |
|---|---|
| A | Many cells forming a cohesive layer |
| B | Few cells forming a loose layer |
| C | Very few large cells |

TABLE 1C

| Grade | Description (Blastocyst) |
|---|---|
| POOR (4) | Early cavitation (e.g., day 6), or morula (e.g., day 5) |
| MEDIOCRE/ | Moderate expansion (e.g., day 6), or |
| FAIR (3) | Early cavitation (e.g., day 5) |
| ADEQUATE (2) | Fully expanded or hatching (e.g., day 6), or |
| | Moderate expansion (e.g., day 5) |
| GOOD (1) | Fully expanded (e.g., day 5), or |
| | Fully hatching (e.g., day 5) |

TABLE 2C

| Grade | Description (ICM) |
|---|---|
| GOOD (A) | High cell number |
| MEDIOCRE/FAIR (B) | Lower cell number |
| POOR (C) | No apparent ICM, or no cells apparent |

TABLE 3C

| Grade | Description (Trophectoderm) |
|---|---|
| GOOD (A) | Good cell-cell adhesion |
| MEDIOCRE/FAIR (B) | Poorer cell-cell attachment |
| POOR (C) | Sparse granular cells, or degenerate |
| | cells in trophectoderm |

Generally, for a transfer and/or for a fertilization/transplantation technique, the healthiest blastocysts are selected, as is understood in the field. This generally includes blastocysts having higher scores for expanding and/or hatching as well as higher scores (Good or A) for the ICM and/or the trophectoderm. Such high grading and/or healthy blastocysts often include ones that are the most advanced. For example, one representative blastocyst suitable and, thereby, selected for such a transfer and/or for a fertilization/transplantation is one that is well expanded on day 6, with a good cell count in the ICM (e.g., greater than 12, or from 12 to 16), and good cell-cell adhesion in the trophectoderm. Using TABLES 1C, 2C and 3C, such a blastocyst would receive a score of 2AA. Another representative blastocyst suitable and, thereby, selected for such a transfer and/or for a fertilization/transplantation is one that is fully expanded on day 5, with a low cell number or very loosely formed ICM, and good cell-cell adhesion in the trophectoderm. Using TABLES 1C, 2C and 3C, such a blastocyst would receive a score of 1BA.

Another representative blastocyst suitable and, thereby, selected for such a transfer and/or for a fertilization/transplantation technique is one that is expanded or is hatching on day 5 (e.g. grade 4 or grade 5, respectively, of Table 1B), has many tightly packed cells in the ICM (e.g., grade A of TABLE 2B), and has a trophectoderm with few cells or a loose epithelium (e.g., grade B of TABLE 3B). Using TABLES 1B, 2B, and 3B, such a blastocyst would receive a score of 4AB, or 5AB, respectively. Another representative blastocyst suitable and, thereby, selected for such transfer and/or for a fertilization/transplantation is one that is partially hatching (e.g. grade 4 of Table 1A), with many tightly packed cells in the inner mass (e.g., grade A of TABLE 2A), and many cells in the trophectoderm (e.g., grade A of TABLE 3A). Using TABLES 1A, 2A, and 3A, such a blastocyst would receive a score of 4AA.

In view of the above, in one or more embodiments, a blastocyst that would be selected for and be considered a viable candidate for transfer and/or for a fertilization/transplantation technique, as is understood in the field (e.g., having higher scores higher scores (Good or A)) will generally not be a candidate as a receiver blastocyst utilized with the systems and methods described herein.

In one or more embodiments, it is discarded blastocysts that may be the receiver blastocyst, for manipulation in the manner with the system and methods described herein.

In one or more embodiments, utilizing any of the grading or scoring systems described herein (or their equivalents, as is understood and/or utilized in the field), blastocysts that are not identified as healthy, and/or do not receive a high enough score as described above, and are discarded (e.g., not suitable for a fertilization/transplantation technique), may be suitable candidates for utilization with the system and methods described herein.

In addition to the other embodiments, described herein, the donor and/or the receiver blastocyst may be a blastocyst that is beginning or has begun a process of expansion, as is it understood in the field, and/or as is described below, including any one or more of TABLES 1A, 1B, 1C, and has a trophectoderm morphology of few cells forming a cohesive epithelium (e.g., grading of B, or scoring of Fair or Mediocre). Discarded blastocysts may still include the zona (enveloping all or at least a majority of the blastocyst).

In some embodiments, a discarded blastocyst may be utilized that does have a good or high trophectoderm score, in addition to blastocyst progression (e.g., as expanding, or expanded), and may be used as a donor blastocyst and/or a receiver blastocyst as described herein. A trophectoderm score of Good, in addition to some blastocyst progression may be used to select a donor blastocyst as described herein. A representative example of such trophectoderm scoring is provided in TABLE 4, which resembles the grading in TABLES 3A and 3B, and is adapted from Thompson S M, et al., 2013, J Assist Reprod Genet, vol. 30, pp. 1577-1581, which is incorporated herein in its entirety.

TABLE 4

| Grade | Description (Trophectoderm) |
|---|---|
| GOOD | Many cells forming a cohesive epithelium |
| FAIR | Few cells forming a cohesive epithelium |
| POOR | Very few cells forming a cohesive epithelium |

In addition to the other embodiments described herein, a receiver blastocyst being manipulated as described herein may be a blastocyst that is expanding, as is understood in the field, and/or as is described below, including any one or more of TABLES 1A, 1B, 1C, and has a trophectoderm morphology of few cells forming a cohesive epithelium (e.g., grading of B, or scoring of Fair or Mediocre).

In addition to the other embodiments described herein, a receiver blastocyst being manipulated as described herein may be a blastocyst that is expanded, as is understood in the field, and/or as is described below, including any one or more of TABLES 1A, 1B, 1C, and has a trophectoderm morphology of few cells forming a cohesive epithelium (e.g., grading of B, or scoring of Fair or Mediocre).

In addition to the other embodiments described herein, a receiver blastocyst being manipulated as described herein may also be a blastocyst that is hatching, as is understood in the field, and/or as is described below, including any one or more of TABLES 1A, 1B, 1C, and has a trophectoderm morphology of few cells forming a cohesive epithelium (e.g., grading of B, or scoring of Fair or Mediocre).

In addition to the other embodiments, described herein, a receiver blastocyst being manipulated as described herein may also be a blastocyst that is beginning or has begun a process of expansion, as is understood in the field, and/or as is described below, including any one or more of TABLES 1A, 1B, 1C, and has a trophectoderm morphology of very few cells a forming a cohesive epithelium and/or very few, unevenly distributed cells (e.g., grading of C, or scoring of Poor).

In addition to the other embodiments, described herein, a receiver blastocyst being manipulated as described herein may also be a blastocyst that is expanded, as is understood in the field, and/or as is described below, including any one or more of TABLES 1A, 1B, 1C, and has a trophectoderm morphology of very few cells a forming a cohesive epithelium and/or very few, unevenly distributed cells (e.g., grading of C, or scoring of Poor).

In addition to the other embodiments, described herein, a receiver blastocyst being manipulated as described herein may also be a blastocyst that is hatching, as is understood in the field, and/or as is described below, including any one or more of TABLES 1A, 1B, 1C, and has a trophectoderm morphology of very few cells a forming a cohesive epithelium and/or very few, unevenly distributed cells (e.g., grading of C, or scoring of Poor).

In addition to the other embodiments, described herein, a receiver blastocyst being manipulated as described herein may also be a blastocyst that is beginning or has begun a process of expansion, as is understood in the field, and/or as is described below, including any one or more of TABLES 1A, 1B, 1C, and has a trophectoderm morphology showing degenerative cells (e.g., grading of D).

In addition to the other embodiments, described herein, a receiver blastocyst being manipulated as described herein may also be a blastocyst that is expanded, as is understood in the field, and/or as is described below, including any one or more of TABLES 1A, 1B, 1C, and has a trophectoderm morphology showing degenerative cells (e.g., grading of D).

In addition to the other embodiments, described herein, a receiver blastocyst being manipulated as described herein may also be a blastocyst that is hatching, as is understood in the field, and/or as is described below, including any one or more of TABLES 1A, 1B, 1C, and has a trophectoderm morphology showing degenerative cells (e.g., grading of D).

FIG. 1B provides illustrative examples of trophectoderm morphologies in accordance with a trophectoderm grading of A, B, C, and D, which generally follows the descriptions provided herein, and/or as is understood in the field, and/or as depicted, for example, in TABLE 3A.

For certain exemplary embodiments described herein, the donor blastocyst will have at least a higher trophectoderm grade and/or better trophectoderm morphology or score and/or a higher number of trophectoderm cells as compared with the receiver blastocyst. For example, a receiver blastocyst may be selected as one that has a trophectoderm grade of any one of C or D, and a donor blastocyst may be selected from one that has a trophectoderm grade of any one of A or B. In some embodiments, a receiver blastocyst as described herein may have a trophectoderm morphology graded as B, which means a donor blastocyst will have a trophectoderm morphology graded as A.

For certain exemplary embodiments described herein, the donor blastocyst will have at least a trophectoderm quality or score of B and/or C.

For certain exemplary embodiments described herein, the donor blastocyst will have at least a trophectoderm quality or score of B and/or C, and an intracellular mass quality of B and/or C.

For certain exemplary embodiments described herein, the donor blastocyst may have a trophectoderm quality or score of A and/or B.

For certain exemplary embodiments described herein, the donor blastocyst will have at least a trophectoderm quality or score of A and/or B, and an intracellular mass quality of B and/or C.

In some embodiments, the receiver blastocyst and the donor blastocyst are discarded blastocyst at about 5 days, or about 6 days, or about 7 days post-fertilization and/or insemination (e.g., about 110 hours post-fertilization and/or insemination, about 120 hours post-fertilization and/or insemination, or about 140 hours post-fertilization and/or insemination, or greater than 140 hours post-fertilization and/or insemination), and have a trophectoderm quality or score of B, and/or C, and an intracellular mass quality of B and/or C. In some embodiments, the receiver blastocyst may have a same or higher ICM quality or score than the donor blastocyst.

Blastocyst manipulation, which includes manipulation of the donor blastocyst and manipulation the receiver blastocyst, is at least one portion of a system described herein, the system comprising at least a microscopic system, a micromanipulation system, a laser system, and a culturing system.

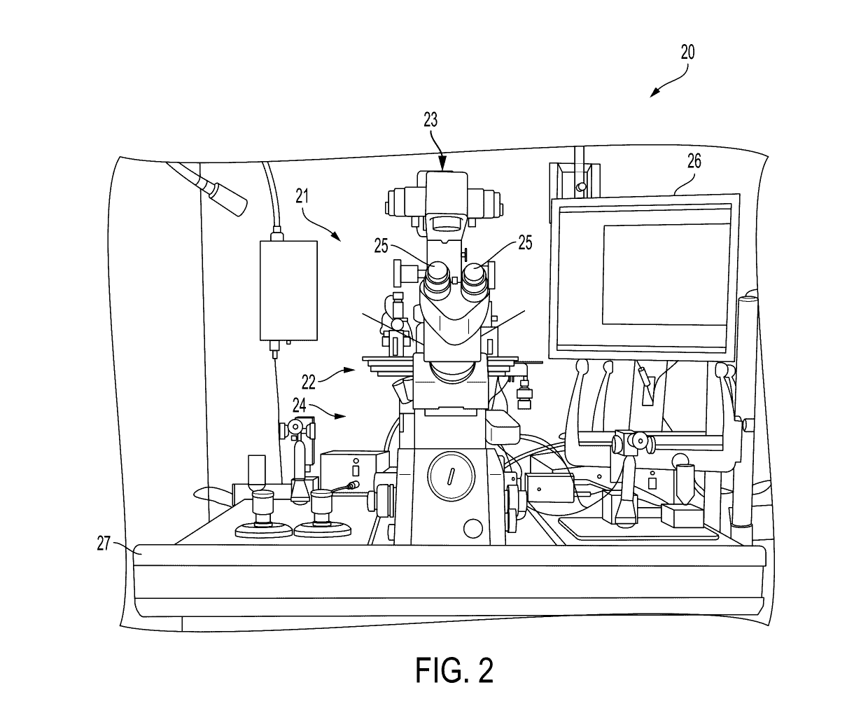

A representative microscopic system 20 for observing blastocysts, for image and/or video capture of the blastocysts, and for assisting in the manipulation of the blastocyst, as will be described in more detail below, is depicted in FIG. 2, showing a microscope 21 with suitable optics therein, and is of the kind known in the art for use with transfer and/or fertilization/insemination/transplantation techniques, such as IVF. In one or more embodiments, the microscope 21 is an inverted phase contrast microscope of the type known and/or utilized in the field disclosed herein. An example of an inverted microscope for use with the systems and methods described herein is a Nikon Eclipse TE2000, or any of the Nikon Eclipse Ti series. Of course, those of skill in the art understand there are many comparable inverted microscopes that may also be utilized. Such a microscope will generally include, in addition to its microscope features: (a) an attachable mechanical and/or motorized stage 22 having low and/or high-speed movement (preferably in x-y and z directions, and which may include heating and cooling options); (b) a digital camera system 23 for video and/or image capture; (c) a light transmission source 24; (d) observation units 25; (e) at least one control unit 26 for at least image and data processing and/or analysis, and for integrating control of the digital camera system 23; (f) associated operating, and processing and/or analytic software, for operation and/or use of the microscope, and any peripherals (e.g., micromanipulators, joysticks, laser, etc.). The microscope may include, and preferably includes, a dedicated Hoffman Modulation Contrast (HMC) system having appropriate objectives and condenser components for obtaining high contrast three-dimensional images of living specimens, which includes the blastocysts described herein, contained in glass or plastic containers or dishes. HMC and its principles are known in the art, having been described by Robert Hoffman in 1975. The microscopic system 20 may be positioned on a platform 27 that provides damping, vibration isolation and/or minimizes vibration, such as, but not limited to a Vibraplane platform from Kinetic Systems.

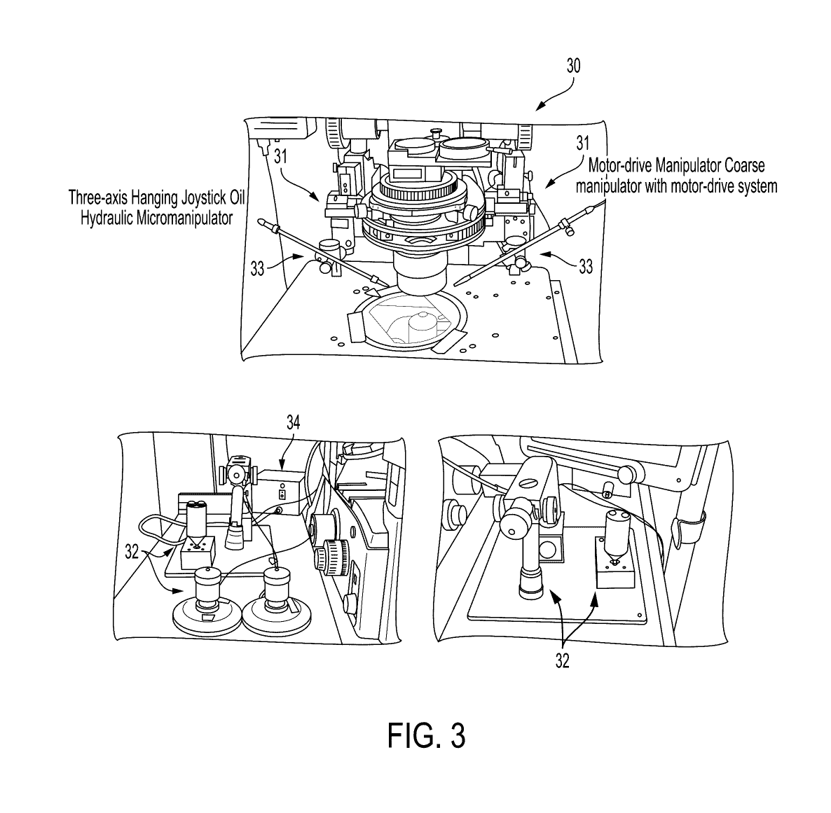

A representative micromanipulation system 30 for assisting in manipulation of the blastocysts is depicted in FIG. 3, showing positioners 31 (which may include three-axis mechanical and/or motorized positioners), joystick micromanipulators 32 (which may include drop-handles and/or universal joints), hydraulic pipette/needle holders 33 (which may be or include one or more double pipette holders and/or universal joints), one or more motor and/or power sources 34, a plurality of connectors, at least one control unit/control box (not shown), and operating software. A microinjector unit (e.g., analog, digital, positive displacement and/or high speed injector, utilizing a pneumatic or gas pressure source, controller, tubing, and/or vacuum) may and is generally included for injecting/extracting/suctioning procedures, which may be preprogrammed for timing, pressure, etc. The micromanipulation system 30 system fits with the inverted microscope of the microscopic system, as depicted in FIG. 3. The micromanipulation system 30 may include ultra-precise piezo motor micromanipulators that provide fine movement with very low drift (˜1 nm/min). An example of a micromanipulation system for use with the systems and methods described herein is a Narishige micromanipulation system from Narishige International Inc. Of course, those of skill in the art understand there are also many comparable micromanipulations systems that may be utilized, and are of the kind known in the art for use with transfer and/or fertilization/insemination/transplantation techniques, such as IVF.

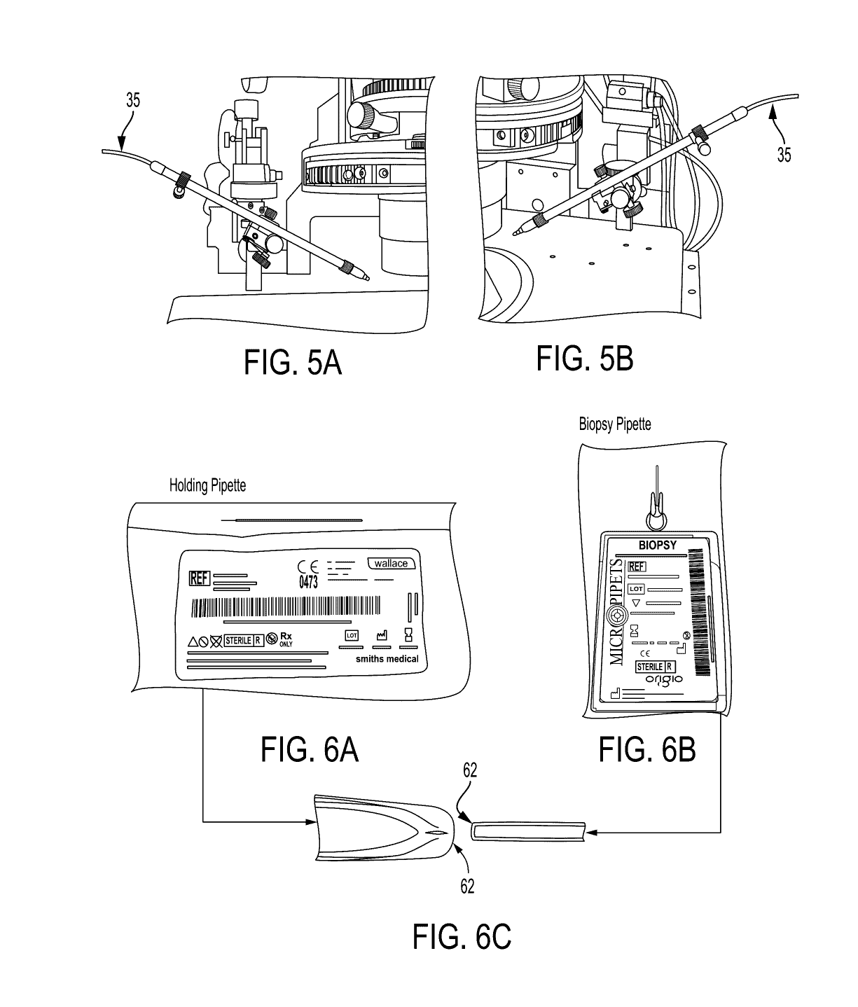

The micromanipulation system further comprises two types of needles and operates utilizing pulsed flow. The first needle is a holding needle, utilized to hold and/or immobilize the blastocyst (FIG. 5A, which further shows a portion of the suctioning system 35 associated with the micromanipulation system). A representative type of holding needle is depicted in FIG. 6A, which comprises a micropipette having an inner diameter of about 15-30 micrometers (or may be 10-30 micrometers, as another example), and an outer diameter of about 120 micrometers (or may be 90 micrometers, or may be 100 micrometers, or may be 130 micrometers, as other examples), having an angle of about 20 degrees, or about 30 degrees (or may be from 0 to 45 degrees, as other examples), and a tip to elbow length of about 0.65 mm. Such a needle, or any suitable type holding needle for the manipulation and holding of a blastocyst as described herein, may be prepared or may be obtained commercially. The size of the blastocyst will help determine the holding needle type and size, including inner and outer diameters. The commercial type of holding needle depicted in FIG. 6A is a WALLACE® holding pipette (last registered with Smiths Medical International Limited, Kent, UK). It is understood in the relevant field that other commercial manufacturers may also be used that provide suitable holding needles for the holding and/or immobilizing of the blastocyst, as described herein.

The second needle utilized with the manipulation described herein is a biopsy needle, as depicted in FIG. 5B, which also shows a portion of the suctioning system 35 associated with the micromanipulation system. A representative type of biopsy needle is depicted in FIG. 6B, which comprises a micropipette generally designed for blastomere biopsy, which may be straight or may be angled (from about 15 degrees or 30 degrees or any angle from about 15 degrees to about 45 degrees), having a flat and polished end (or may have a beveled and polished end), and may have an inner diameter of about 33 to 37 micrometers (or may be 28-32 micrometers, or may be 38-42 micrometers, as other examples). Such a needle, or any suitable type biopsy needle for the manipulation and/or disruption of a blastocyst as described herein, may be prepared or may be obtained commercially. The size of the blastocyst will help determine the biopsy needle type and size, including inner diameter. The commercial type of holding needle depicted in FIG. 6B is an Origio Humagen blastomere biopsy micropipet (a Denmark company). It is understood in the relevant field that other commercial manufacturers may be used that provide suitable biopsy needles for the blastocyst, as described herein. FIG. 6C illustrates, in high resolution, an end of an injection holder for a holding needle of the type described herein and with FIG. 6A (left side), and a biopsy needle (right side) of the type described herein and with FIG. 6B, in which the biopsy needle in the image is a 1 mm (outer diameter) micropipette with a flat and polished end, and each of the biopsy needle and the holding needle have a working end 62.

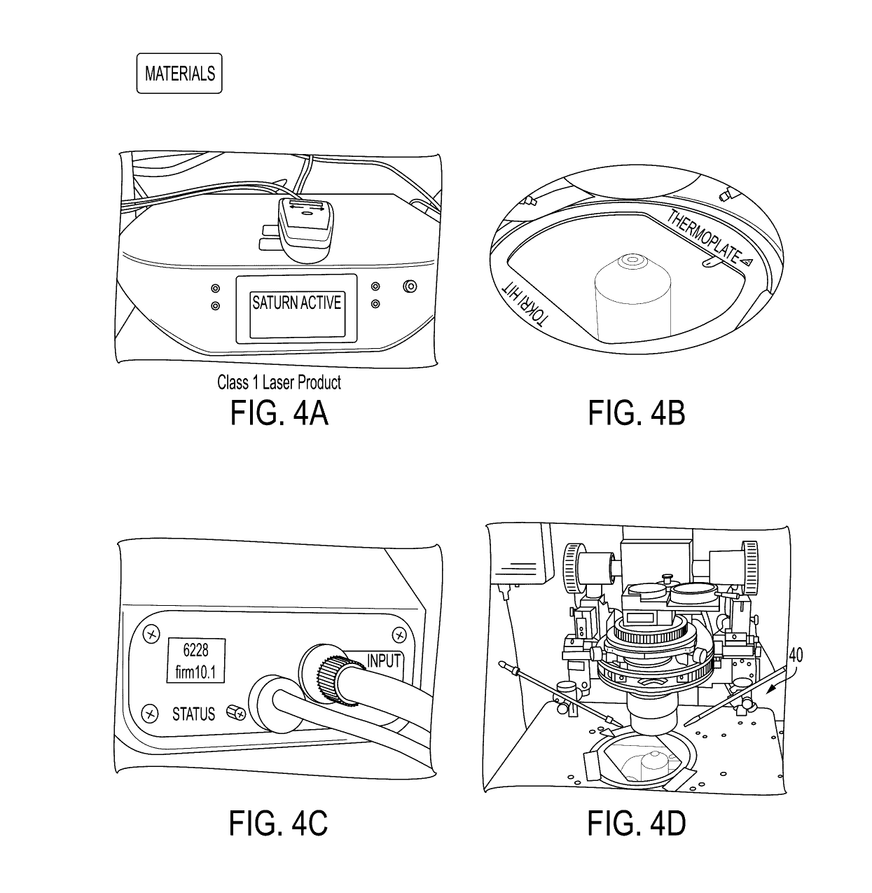

A representative laser system 40 for manipulation and/or precise ablation of a portion of the blastocyst as described herein is depicted in FIG. 4A-4D, which utilizes a high power laser that applies less energy for achieving an ablation or hole size, as compared with lower power laser systems. The laser system fits with the inverted microscope of the microscopic system, and comprises an ablation laser depicted best in FIGS. 4B, 4D, which may be a solid state diode, and may be computer controlled. A suitable laser diode (or semiconductor laser diode) may provide light at up to about or greater than about 1400 nm (e.g., about 800 to about 1400 nm, or about 1400 nm to about 2000 nm), and at a pulse length in a range from about 0.005 to 2.0 milliseconds (5 to 2000 microseconds). The ablation and laser may be provided from a fiber optic, collimated by an achromatic collimator. The laser system may further comprise a red pilot beam for spotting or providing a target, which may be provided by a red diode (e.g., emitting light in the red wavelength, such as from 600-650 nm, which may use a same optical path and fiber optic as the ablation laser). The ablation laser and pilot laser will generally be a Class 1 laser product as defined by International laser safety standards. The laser system additionally includes an objective (generally 20×, or 40×), at least one control unit (FIGS. 4A, 4C), operating software, and may include robotics for laser control. The laser system may further include a mirror module (for transmitting at higher wavelengths that are greater or much greater than 650 nm), and a motor module, and filter for the red light (infrared filter). The laser system may further include a one click mouse and/or foot pedal for laser function and/or image capture and/or video recording. The laser system may be combined with or may include measurement and/or imaging software. A representative laser system is a Saturn 5 Active Laser System, from RI (Research Instruments) Life Sciences. A preferred laser system is one in which the laser is moveable without having to move the blastocyst. The laser power may be about 400 mW, as a representative example. The laser pulse width may be about 0.606 milliseconds, as a representative example. A hole size generated by such a laser may be about 5 micrometers to about 12 micrometers. A hole size may be less than 10 micrometers.

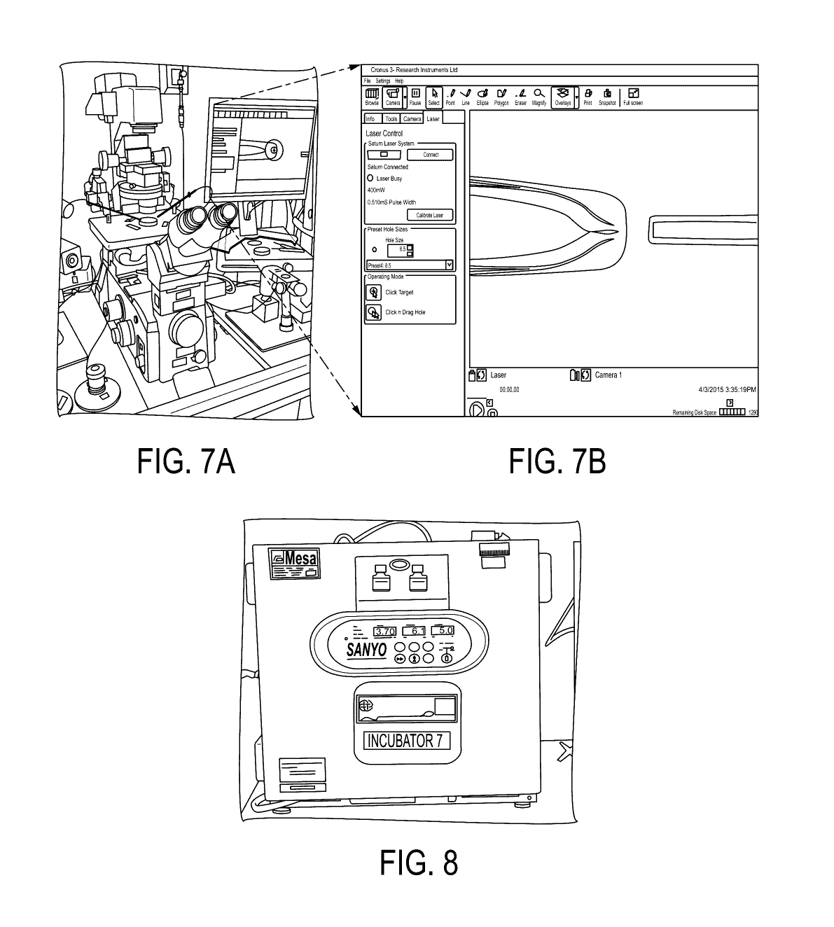

The microscopic system, the micromanipulator system, and the laser system may be integrated and/or coordinated through a computing device and associated software, such as one that includes a digital video recorder module, an image capture module, an analysis module, and various reporting functions, in addition to one or more operating functions. The computing device and associated software may be further integrated and/or in communication with one or more databases and/or one or more patient records. The computing device and associated software, when integrated with and/or accessible with the microscopic system, and the micromanipulator system, will allow for at least visualization of manipulation (micromanipulation) of the blastocyst, as well as control of all or portions of the manipulation, capture of data associated with the manipulation, and analysis thereof, as depicted in FIG. 7A. The computing device and associated software, when further integrated with the laser system, will allow for laser control during micromanipulation, as depicted in FIG. 7B. A representative software for use with the system and the computing device described herein is CRONUS 3™ from Research Instruments (Denmark). It is understood in the relevant field that other software may be used with the system and the computing device described. Such software may also be utilized for blastocyst grading and/or scoring, by including and utilizing a customizable grading and/or scoring module.

Any computing device and associated software provided with the system described herein will provide at least digital data, which may be stored, and/or analyzed and/or captured and/or transmitted, utilizing binary data, digital images, digital video, tables, charts, etc. Any of the data collected by the system described herein may be stored in a storage device, which may or may not be the same storage device storing the software, and may or may not be the same storage device storing information and/or data about a patient. In addition, any of the data described herein may be processed and/or accessed by one or more suitable computing devices, such as the type having a central processing unit comprising logic, control circuitry, and memory (e.g., as volatile memory, cache memory) in addition to a storage space or memory (e.g., as non-volatile memory). The computing device may be any form (e.g., desktop computer form, portable computing form, smartphone, personal computer, laptop, electronic tablet device, global positioning system (GPS) receiver, portable media player, personal digital assistant (PDAs), and/or network access device), including any processing device capable of receiving and transmitting data. The computing system will require or will be coupled to a screen for visualization. The screen may be a touch screen. The same computing device, or a separate computing device may be utilized to process and/or analyze and/or communicate the data. Similarly, the same computing device or a separate device may be used to store the data, which may be stored on a computer (machine) readable medium, in a computer (machine) readable form. A computer-readable medium may include any medium that participates in providing instructions to one or more processors for execution. Such a medium may take many forms including, but not limited to, nonvolatile, volatile, and transmission media. Nonvolatile media includes, for example, flash memory, or optical or magnetic disks. Volatile media includes static or dynamic memory, such as cache memory or certain RAM. Transmission media may include coaxial cables, copper wire, fiber optic lines, and wires arranged in a bus. Transmission media may also take the form of electromagnetic, radio frequency, acoustic, or light waves, such as those generated during data communications, such as radio wave, and/or infrared data communications.

The computing device associated with at least one of and/or integrated with one or more of the microscopic system, the micromanipulator system, and the laser system, will include at least one processor, and may or may not include a telephonic portion (and associated radios/receivers), and may or may not have touch screen display for accessing some or all of the data, and/or a keyboard for accessing some or all of the data. A user should be able to interface with at least the computing device. Suitable computer operating systems software for the computing device, in addition to the software described above (the software associated with the microscopic system, the micromanipulator system, and/or the laser system), may be from a Microsoft WINDOWS® family from Microsoft Corporation (e.g., Windows 95, 98, Me, Windows NT, Windows 2000, Windows XP, Windows XP x64 Edition, Windows Vista, Windows 7, Windows 8, Windows CE, Windows Mobile, Windows RT), or from any one of Symbian OS, Tizen, Linux, HP-UX, UNIX, Sun OS, Solaris, Mac OS X, Apple iOS, Android, Alpha OS, AIX, IRIX32, or IRIX64, as non-limiting examples. The software associated with the microscopic system, the micromanipulator system, and/or the laser system may be an independent application with data input and data display modules. Such software will be compatible with and/or may be integrated with some of all of the computer operating systems software for the computing device.

The software associated with the microscopic system, the micromanipulator system, and/or the laser system (e.g., a binary, machine-executable version) is stored on at least the computing device in memory (e.g., RAM and/or cache memory), and/or is stored on one or more alternative storage units (e.g., hard disk, magnetic disk, tape, or CD-ROM, one or more servers) and accessible by the computing device. As a further example, code or the software may be accessible and/or transmitted via wires, radio waves, cloud computing, and/or through a network such as the Internet. In operation, a system described herein may include one or more computer devices used to execute the software accessible by and/or associated with the one or more computing devices, which will be involved in implementing one or more of the embodiments and/or steps of the embodiments described herein.

The system described herein may further includes subsystems such as: one or more central processors, system memory (volatile and/or nonvolatile), input/output (I/O) controller, display adapter, serial or universal serial bus (USB) port, one or more network interfaces, one or more servers, firewall, and camera, and/or speaker, in addition to the microscopic system, the micromanipulator system, and the laser system. Additional or fewer subsystems may be included. The system may be a multiprocessor system. The processor of the computing device may comprise and/or be associated with multiple processors, or a multicore processor, which may permit parallel processing of information described herein. Other configurations of subsystems suitable for use with the embodiments described herein will be readily apparent to one of ordinary skill in the art.

In one or more embodiments, the system described herein, comprising the one or more computing devices associated with at least one of and/or integrated with one or more of the microscopic system, the micromanipulator system, and the laser system, as described herein, may be connected to a network. The system may interface with other computers using the network, including one or more computers that capture at least some of the data associated with the blastocysts. The network may be an intranet, internet, or the Internet, as examples. The network may be a wired network, telephone network, packet network, and/or optical network (e.g., using optical fiber). In addition, or as an alternative, the network may be a wireless network. As an example, the network may be a wireless network using a protocol such as Wi-Fi (under IEEE standard, e.g., 802.11, 802.11a, 802.11b, 802.11e, 802.11g, 802.11i, 802.11n, 802.11ac, and 802.11ad), and/or a near field communication (NFC), and/or a radio-frequency identification (RFID), and/or a mobile and/or cellular wireless communication (e.g., 2G, 3G, 4G, 3GPP LTE, WiMAX, LTE, LTE Advanced, Flash-OFDM, HIPERMAN, iBurst, EDGE Evolution, UMTS, UMTS-TDD, 1×RDD, and EV-DO).

In at least one embodiment, the system described herein will include a workstation operable with and executing an application, which may be a local application, or one accessible through the Internet, via a web browser. The web browser provides web pages and content thereon in suitable formats (e.g., HTML, XML, text, PDF, postscript). The web browser allows data to be downloaded from the web pages, and may be used to upload other data, which may be accessible to other parts of the system, such as by hypertext transfer protocol (HTTP) for transferring files on the web. Examples of web browsers include, but are not limited to Internet Explorer (provided by Microsoft Corporation), Firefox (provided by Mozilla), Chrome (provided by Google), Safari (provided by Apple, Inc.). The local application may be on the hard drive or a disk, as examples. In some embodiments, the local application may be updated (e.g., periodically or on demand) via a direct internet upgrade linking mechanism, or through an applications storehouse (e.g., Apple iTunes and App store, Google Play store, Windows Phone App store, etc.). In some embodiments, the web-based application may be associated with a cloud infrastructure, relying on a virtual desktop, and having accessible software associated with the system stored remotely on one or more remote storage units. The cloud infrastructure may comprise any of a number of suitable platforms (e.g., software as a service (SaaS), platform as a service (PaaS), infrastructure as a service (IaaS), combinations, and/or improvements thereof. Examples of cloud computing services are Google Drive, Apple iCloud, Microsoft OneDrive, Microsoft Office Online, Amazon Cloud Drive, that may be adapted to a system described herein.

For the system described herein, one or more servers may be associated with the one or more computing devices that are associated with one or more of the microscopic system, the micromanipulator system, and the laser system, as described herein described herein. The one or more servers may include a client system operably coupled with and/or in communication with the network (e.g., via an access provider, and/or a server system). The client system(s) typically request information from the server system, which provides the information. In some embodiments, a computer system may act as both a client system and a server system depending on whether the computer system is requesting or providing information, and/or may act as a stand-alone computer system.

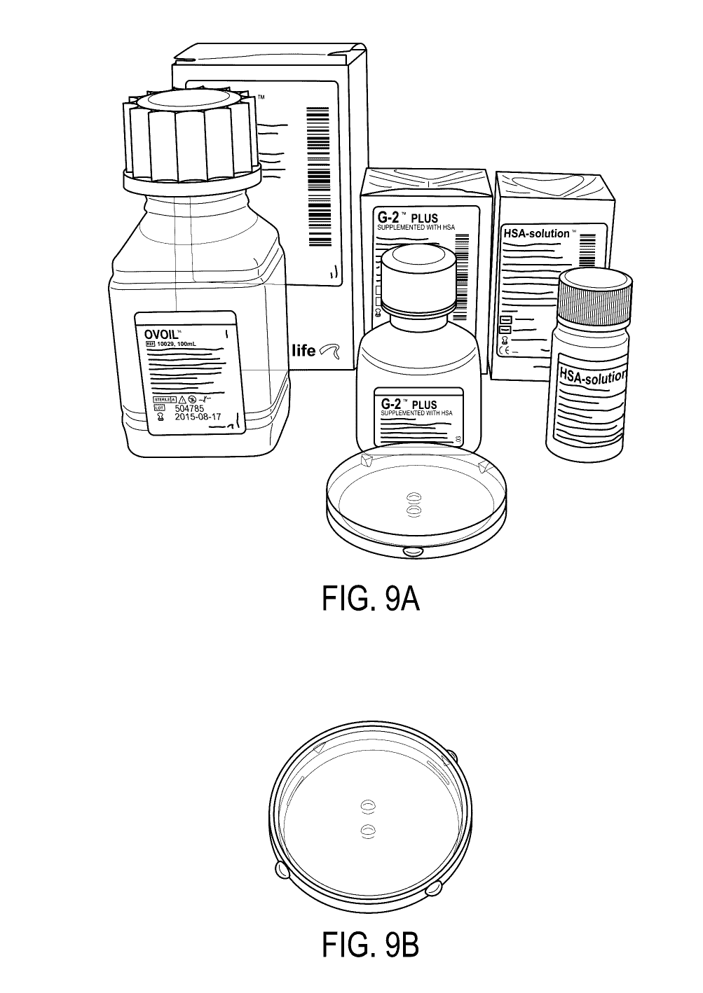

The system described herein, comprising the microscopic system, the micromanipulator system, and the laser system, further comprises a culturing system, such as depicted in FIGS. 8, 9A, and 9B. FIG. 8 shows a representative multi-gas incubator. The incubator may include an air jacket with precise control of gas levels (e.g., CO₂, O₂) and/or humidity, and will include one or more heaters. FIGS. 9A and 9B show additional representative components of the culturing system, including: (a) human serum albumin solution, which provides the albumin in an amount that is about 100 mg/mL; (b) a bicarbonate buffered culturing medium containing human serum albumin, hyaluronan, amino acids, and gentamycin; and (c) a light paraffin oil having a low peroxide level; (d) a culture dish. The culture dish as depicted in FIGS. 9A and 9B, contain an independent droplet of culturing or maintenance medium, which may be under a paraffin oil overlay.

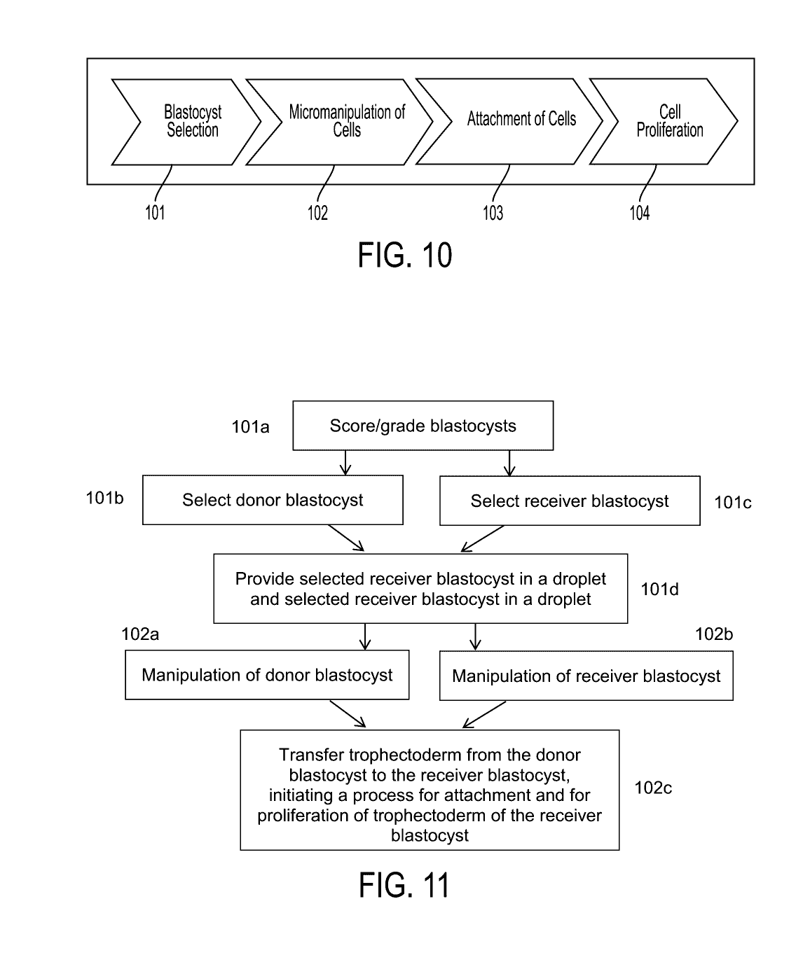

Systems, methods, and cellular compositions generated therefrom, are further depicted in whole or in part in at least FIGS. 10-14. FIG. 10 outlines, in general, various features and/or principles of operation, including blastocyst selection (block 101), micromanipulation (block 102), cell attachment (block 103), and cell proliferation (block 104).

Blastocyst selection, as described herein and above, utilizing at least a portion of the system described herein, comprising at least the microscopic system, the micromanipulation system, the culturing system, and one or more computing devices, includes a method comprising selection of two blastocysts (block 101, FIG. 10).

Blastocyst manipulation, which includes micromanipulation of the two selected blastocysts (block 102, FIG. 10), utilizing the system described herein, comprising at least the microscopic system, the micromanipulation system, the laser system, the culturing system, and one or more computing devices, includes a method comprising sequential and/or simultaneous micromanipulation of the two selected blastocyst (block 102, FIG. 10).

Together, the blastocyst selection, as described herein, and the micromanipulation of selected blastocysts, as described herein, deliver benefits to one of the selected blastocysts, which includes transfer of cells to the one selected blastocyst, attachment of cells in the one selected blastocyst (block 103, FIG. 10), and proliferation of cells in the one selected blastocyst (block 104, FIG. 10). Such benefits further provide one of the selected blastocysts with an improved outcome for clinical pregnancy and birth. Such benefits allow for rescue of one of the selected blastocysts that was otherwise discarded, and/or considered not viable for transfer, and/or considered not suitable for utilization in a fertilization/insemination/transplantation technique (as is understood in the relevant art).

Referring again to the blastocyst selection, this feature as described herein includes identifying blastocysts collected at a period of time after fertilization and/or insemination. The period of time may be three days, or may be four days, or may be five day, or may be six days, or may be seven days. The identifying will include scoring and/or grading the blastocysts as described above.

As further depicted in FIG. 11, blastocyst selection is based on scoring and/or grading (block 101a). In one or more embodiments, a plurality of blastocysts are scored and/or graded. In one or more embodiments, two or more blastocysts are scored and/or graded.

Continuing with FIG. 11, micromanipulation includes a first manipulation (block 102a) of at least a first donor blastocyst that has been selected (block 101b), in which the selection is from and/or based on scoring and/or grading as described herein (block 101a). Micromanipulation further includes a second manipulation (block 102b) of at least a first receiver blastocyst that has been selected (block 101c), in which the selection is from and/or based on scoring and/or grading as described herein (block 101a). In some embodiments, the first donor blastocyst and the first receiver blastocyst are identified and scored/graded within a same or similar time frame (e.g., close in time and in series, and/or close in time and substantially in parallel). In one or more embodiments, the first donor blastocyst and the first receiver blastocyst will be ones collected at a same or substantially a same fertilization and/or insemination period, or will be ones collected in a similar period of time after fertilization and/or insemination, and will be from a same fertilization and/or insemination. In additional embodiments, the first donor blastocyst and the first receiver blastocyst are identified and scored/graded within a same or similar time frame (e.g., close in series, and/or in parallel), and the first donor blastocyst and the first receiver blastocyst will be ones collected from a different period of time after fertilization and/or insemination. In one or more embodiments, the grading and/or scoring utilized to select the first donor blastocyst is the same, substantially the same, or similar, as the grading and/or scoring utilized to select the first receiver blastocyst. In one or more embodiments, more than one donor blastocyst may be selected. In one or more embodiments, more than one receiver blastocyst may be selected.

Each donor blastocyst selected in block 101b will have a scoring and/or grading that is at least the same or about the same as the receiver blastocyst that is selected. In one or more embodiments, and in some preferred embodiments, each donor blastocyst selected in block 101b will have a scoring and/or grading that is higher or better than the receiver blastocyst that is selected, in which selection is in any manner described previously. Similarly, in some embodiments, each receiver blastocyst selected in block 101c will have will have a scoring and/or grading that is at least the same or about the same as the receiver blastocyst that is selected. In one or more embodiments, and in some preferred embodiments, each receiver blastocyst selected in block 101c will have a scoring and/or grading that is lower or less than the donor blastocyst that is selected, in which selection is in any manner described previously. Scoring and/or grading of the first donor blastocyst and of the first receiver blastocyst will at least include a scoring and/or grading of the trophectoderm quality and/or the number of trophectoderm cells in the trophectoderm. Accordingly, the first donor blastocyst selected will always have a higher or better (e.g., grade and/or score) of the trophectoderm, in quality and/or number of trophectoderm cells. Scoring and/or grading of the first receiver blastocyst may include a scoring and/or grading of the trophectoderm quality and/or the number of trophectoderm cells in the trophectoderm, and a scoring and/or grading of the ICM.

Upon selection of the first donor blastocyst and the first receiver blastocyst, and shortly and/or immediately after selection, such blastocysts are prepared for manipulation (block 101d). Preparation may include positioning both the first donor blastocyst and the first receiver blastocyst in a same droplet. The droplet, as described herein, may be prepared from a portion of the culturing system described herein. The droplet is formed from one or two or a plurality of independent droplets, positioned and maintained in a same location on a suitable manipulation container (such as a glass or plastic petri dish, one suitable in the field, such as, but not limited to, the representative container depicted in FIG. 8). Generally, the droplet is comprised of a suitable maintenance medium containing one or more proteins and/or essential amino acids, in which the medium may and is preferably further supplemented with an additional amount of protein, from 10 to 30 wt. % protein, or about 20 wt. % protein). A suitable and representative maintenance medium useful for the droplet is Quinns ADVANTAGE™ Protein Plus media, from Origio, Sweden, which is a modification of human tubal fluid described by Quinn et al., in 1984, Fertil. Steril. 41:202, and in 1985, Fertil. Steril. 44:493, and may include some additional amino acids, taurine, sodium citrate, minimal essential medium vitamins, and calcium lactate. The droplet may further comprise an oil (e.g., paraffin) overlay. Generally, the droplet has been formed well in advance (e.g., pre-incubated, at least or about one hour before, and/or overnight), and is at a temperature suitable for living tissue (e.g., at or about 37 degrees Centigrade). In one or more embodiments, the first receiver blastocyst and the first donor blastocyst are positioned in different droplets. Preferably all droplets and blastocysts are on a same manipulation container. In one or more embodiments, the manipulation container having the one or more droplets is provided on a heated stage of a microscope, in which the stage is heated to a temperature suitable for a living tissue (e.g., at or about 37 degrees Centigrade, or from about 25 to about 40 degrees Centigrade).

Selection and preparation of the selected donor blastocyst and the selected receiver blastocyst may utilize the same system utilized for manipulation of these blastocysts, or may utilize a different micromanipulation system, such as at a different and/or neighboring station.

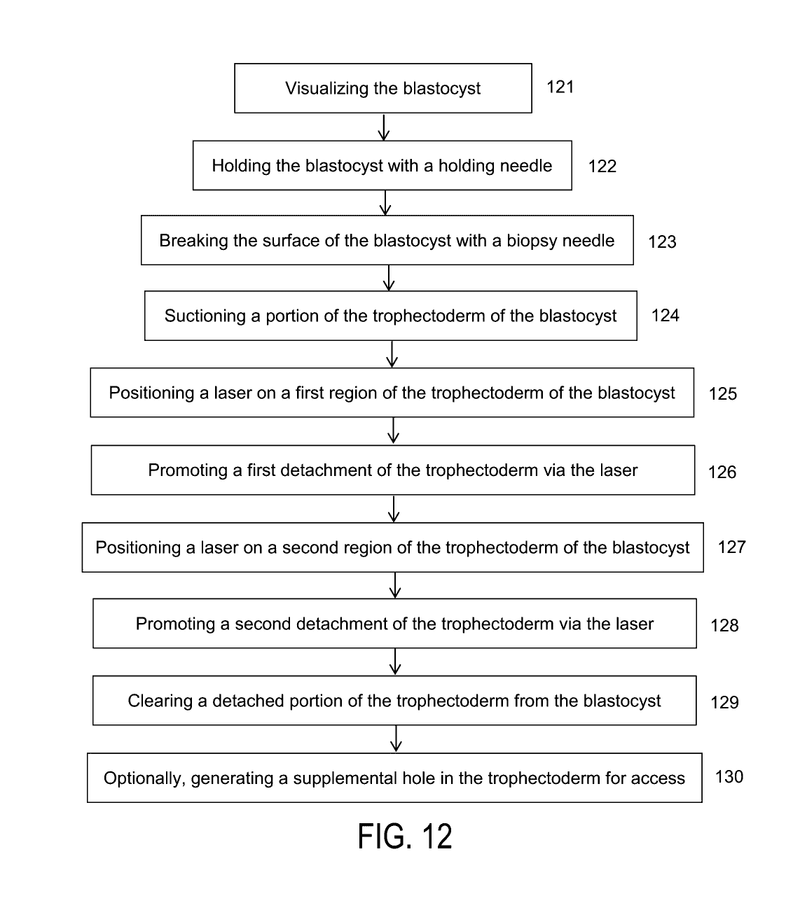

The manipulation of blastocysts includes a first manipulation of the first donor blastocyst (block 102a) selected in accordance with block 101a, and a second manipulation of the first receiver blastocyst (block 102b) selected in accordance with block 101b. Generally, block 102a precedes block 102b. The manipulation of blastocysts is outlined in FIG. 12.

For manipulation of the first donor blastocyst in the first manipulation, the container containing the first donor blastocyst (which may also contain the first receiver blastocyst) are positioned for visualization of at least the first donor blastocyst by the microscopic system described herein, which further comprises and is integrated with the micromanipulation system, and the laser system (block 121, FIG. 12). Visualization will include the proper magnification (e.g., 20X), and camera positioning, and may further comprise visualization of at least the first donor blastocyst on a control unit, or on a visualization screen of a computing device associated, coupled, and in communication with the system.

Manipulation of the first donor blastocyst in the first manipulation further comprises lowering and grasping gently the first donor blastocyst with a holding needle of the type described herein of the micromanipulation system described herein (block 122, FIG. 12). The first donor blastocyst may be held at a portion of the outer surface at or near the ICM. The first donor blastocyst may be held by a light suction of a suction system associated with the holding needle. Holding of the first donor blastocyst at or near the ICM prevents disturbance and/or ablation of the ICM of the first donor blastocyst. In one or more embodiments, disturbance and/or ablation of the ICM of the first donor blastocyst is avoided.

Thereafter, manipulation includes breaking a surface of the first donor blastocyst (block 123, FIG. 12). Breaking the surface may comprise providing the biopsy needle and the zona pellucida of the first donor blastocyst in a same visual field. Breaking the surface may comprise initially lowering the biopsy needle until it breaks a surface of the overlay oil of the droplet, and thereafter positioning the biopsy needle in the same visual field as the first donor blastocyst. Breaking the surface may comprise equilibrating the biopsy needle with the maintenance media of the droplet, after the biopsy needle has broken the surface of the overlay oil.

Once the surface of the first donor blastocyst has been broken, manipulation of the first donor blastocyst in the first manipulation will comprise suctioning a plurality of trophectoderm cells from the trophectoderm of the first donor blastocyst (block 124, FIG. 12). The plurality of trophectoderm cells may comprise more than two trophectoderm cells, or at or about three to five trophectoderm cells, or up to about 10 trophectoderm cells, or greater than 10 trophectoderm cells, or from about three to about 10 trophectoderm cells. The plurality of trophectoderm cells may comprise additional living or organic material of the blastocyst, which may include a portion of the zona pellucida and/or tight junctions associated with and formed between neighboring trophectoderm cells of the plurality of trophectoderm cells. The suctioning of the plurality of trophectoderm cells may include clearing some or all of the plurality of trophectoderm cells of the first donor blastocyst from the zona pellucida of the first donor blastocyst. The suctioning of the plurality of trophectoderm cells may include holding at least a portion of the plurality of trophectoderm cells in the biopsy needle. The suctioning of the plurality of trophectoderm cells may include extending at least a portion of the plurality of trophectoderm cells from the first blastocyst, as well as the material associated with the plurality of trophectoderm cells, so that there is an extended portion of the plurality of trophectoderm cells away from the first donor blastocyst. In some embodiments, the plurality of trophectoderm cells includes the extended portion. In some embodiments, the extended portion includes at least a portion of the plurality of trophectoderm cells being held in the biopsy needle, and at least a portion of the plurality of trophectoderm cells being adjacent the portion that is held in the biopsy needle. The suctioning and/or extending of the plurality of trophectoderm cells may include using joystick(s) associated with the biopsy needle, and using a suctioning control associated with the biopsy needle.

Manipulation of the donor blastocysts of the first manipulation further comprises a first positioning of the laser of the laser system on at least a first region of at least one trophectoderm cell that forms a part of the plurality of trophectoderm cells of the first donor blastocyst (block 125, FIG. 12). The first positioning of the laser may comprise aligning the laser along one of the trophectoderm cells of the first region. The first positioning of the laser may comprise positioning of the pilot laser on cells that are held in the biopsy needle. The first positioning of the laser may comprise aligning the laser, which may be the pilot laser, between one trophectoderm cell and a next trophectoderm cell that is immediately neighboring. The first positioning of the laser, which may be the pilot laser, may comprise positioning the laser on one or more cells in (or adjacent, or near) the extended portion of the plurality of trophectoderm cells. The first positioning of the laser does not usually include positioning the laser on cells that are held in the biopsy needle. In one or more embodiments, aligning is associated with a heating of the first donor blastocyst, which may assist in the detachment (and/or cutting) of a portion of the first region.

Upon or after the first positioning of the laser on the one or more of the trophectoderm cells of the first region, the laser is activated. Generally, activation of the laser is a pulsing of the laser. In one or more embodiments, activation of the laser may initiate an initial collapse of the donor blastocyst. In one or more embodiments, activation of the laser may initiate an initial collapse of the donor blastocyst followed by a re-expansion of the first donor blastocyst. In one or more embodiments, activation of the laser is accompanied by a detachment of a trophectoderm cell and/or detachment between trophectoderm cells of the first region (block 126, FIG. 12). In one or more embodiments, activation of the laser is accompanied by ablation of one, and preferably only one, trophectoderm cell in the first region. In one or more embodiments, a second activation of the laser may follow to ensure detachment of the trophectoderm cell and/or between trophectoderm cells of the first region. In one or more embodiments, detachment of the first region is facilitated by one or more of suction of at least the portion of the plurality of trophectoderm cells being held by the biopsy needle. In one or more embodiments, detachment of the first region is facilitated by stretching of at least the portion of the plurality of trophectoderm cells being held by the biopsy needle. In one or more embodiments, laser activation is associated with a heating of the first donor blastocyst when detaching and/or cutting a portion of the first region.

Upon or after detachment of the trophectoderm cell and/or between trophectoderm cells of the first region, a second positioning of the laser is provided on a second region of at least one trophectoderm cell that forms a part of the plurality of trophectoderm cells of the first donor blastocyst (block 127, FIG. 12). This second region is spaced apart from the first region. In some embodiments, from the first region to the second region there are at least two or more trophectoderm cells, or at or about three to five trophectoderm cells, or up to about 10 trophectoderm cells, or greater than 10 trophectoderm cells, or from about three to about 10 trophectoderm cells, or any number of trophectoderm cells in a range therebetween.

The second positioning of the laser may comprise aligning the laser along one of the trophectoderm cells of the second region. The second positioning of the laser may comprise positioning of the pilot laser on one or more trophectoderm cells of the second region. The second positioning of the laser on the second region may comprise aligning the laser, which may be the pilot laser, between one trophectoderm cell and a next trophectoderm cell that is immediately neighboring. The second positioning of the laser may comprise positioning the laser, which may be the pilot laser, on one or more cells in (or adjacent, or near) the extended portion of the plurality of trophectoderm cells. The second positioning of the laser does not usually include positioning the laser on cells that are held in the biopsy needle. In one or more embodiments, second positioning, which may include use of the pilot laser, is associated with a heating of the first donor blastocyst and/or heating of the second region, which may assist in the detachment (and/or cutting) of a portion of the second region.

Upon or after the second positioning of the laser on the one or more of the trophectoderm cells of the second region, the laser is activated. Generally, activation of the laser is activation of the ablation laser. Generally, activation of the laser is a pulsing of the laser. In one or more embodiments, activation of the laser may initiate a collapse of the donor blastocyst. In one or more embodiments, activation of the laser may initiate a collapse of the first donor blastocyst followed by a re-expansion of the donor blastocyst. In one or more embodiments, activation of the laser is accompanied by a detachment of a trophectoderm cell and/or detachment between trophectoderm cells of the second region (block 128, FIG. 12). In one or more embodiments, activation of the laser is accompanied by ablation of one, and preferably only one, trophectoderm cell in the second region. In one or more embodiments, a second activation of the laser may follow to ensure detachment of the trophectoderm cell and/or between trophectoderm cells of the second region. In one or more embodiments, detachment of the second region is facilitated by one or more of a light suction of at least the portion of the plurality of trophectoderm cells that may be held by the biopsy needle. In one or more embodiments, detachment of the second region is facilitated by stretching of at least the portion of the plurality of trophectoderm cells that may be held by the biopsy needle. Following detachment of the second region, all or a portion of a detached piece comprising some or all of the plurality of trophectoderm cells (ones initially manipulated in the first manipulation) may enter the biopsy needle. In some embodiments, the detached piece is expelled from the biopsy needle by appropriate pressure applied to the biopsy needle, and remains in the droplet (e.g., in the maintenance fluid or medium comprising at or about 20 wt. % protein, or from about 10 to about 30 wt. % protein), near the first donor blastocyst (block 129, FIG. 12). In some embodiments, the detached piece is expelled from the biopsy needle by appropriate pressure applied to the biopsy needle, and is maintained in a maintenance fluid (e.g., comprising at or about 20 wt. % protein, or from about 10 to about 30 wt. % protein) in another, and appropriately sized, needle of the micromanipulation system. In some embodiments, the detached piece from the first donor blastocyst comprises healthy and/or good trophectoderm cells with good tight junctions. The detached piece from the first donor blastocyst comprises donor trophectoderm cells from the donor blastocyst. In one or more embodiments, the detached piece from the first donor blastocyst does not contain any cells from the ICM of the donor blastocyst. In one or more embodiments, donor cells in the detached piece are not (and/or do not include) cells of the ICM.

First and second positioning of a laser, which may be a first laser, in a laser system as described herein, and activation of the laser, which may be a second laser, of the laser system as described herein are further illustrated in FIGS. 14A to 14G. FIG. 14A shows the first positioning, illustrated in the figure by the downward arrow, in which positioning by a red pilot laser is on a portion of a trophectoderm cell at a first region of a portion of the trophectoderm of a donor blastocyst, in which the portion of the trophectoderm was extended from the zona pellucida of the donor blastocyst. FIGS. 14B to 14D illustrate an extending or lengthening and thinning of the portion of the trophectoderm by a mild suctioning of the biopsy needle. FIGS. 14E to 14G illustrate the second positioning, illustrated in the figures by the upward arrow, in which this positioning by the red pilot laser is on another portion at a second region of the portion of the trophectoderm of the donor blastocyst, the second positioning made along the extending or lengthening or thinning section of the portion of the trophectoderm. In FIGS. 14E and 14F, the extending or lengthening and thinning of the portion of the trophectoderm was continued. FIG. 14G illustrated a detached end of the portion of the trophectoderm after laser activation of an ablation laser that resulted in detachment. The detached piece is not visible in FIG. 14G.

Upon or after complete detachment of the detached piece, manipulation of the first receiver blastocyst by a second manipulation is begun (e.g., block 102b, FIG. 11). In some embodiments, the second manipulation is begun immediately after completion of the first manipulation (e.g., immediately after complete detachment of the detached piece from the first donor blastocyst). Manipulation of the first receiver blastocyst, performed by a second manipulation, may include substantially the same steps and/or similar steps as outlined with the first manipulation, and as depicted in FIG. 12.

For example, the second manipulation of the first receiver blastocyst may be performed in the same droplet or in a different droplet. Generally, a same holding needle, or a second or separate holding needle is utilized to hold and/or grasp the first receiver blastocyst (block 122, FIG. 12). In one or more embodiments, the first receiver blastocyst is held at or near the ICM. This prevents disturbance and/or ablation of the ICM of the first receiver blastocyst. In one or more embodiments, disturbance and/or ablation of the ICM of the first receiver blastocyst is avoided. The first receiver blastocyst and/or the biopsy needle may then be manipulated (e.g., via fine controls, one or more joysticks) in order to include a least a portion of the blastocyst in a same visualization field as the biopsy needle. Preferably, at least a portion of the blastocyst, including a portion of the zona pellucida is in the same visualization field as the working end of the biopsy needle (block 121, FIG. 12). The visualization field, at an appropriate magnification (e.g., 20X), may be that of the microscope and/or a visualization field on a control unit and/or a visualization screen associated with a computing device. Once or after the first receiver blastocyst is held by the holding needle, the biopsy needle is equilibrated with a maintenance-type media and/or a similar biopsy media, such a one suitable for a blastocyst, or one utilized for the droplet. In one or more embodiments, the media for equilibration is a media that further comprises addition protein, which is at or about 20 wt. % protein, or from about 10 to about 30 wt. % protein. Thereafter, the biopsy needle is positioned on the surface of the first receiver blastocyst. In one or more embodiments a surface of the first receiver blastocyst is broken (e.g., ablated and/or removed, as depicted in block 123, FIG. 12). The breaking of the surface may or may not include ablation and/or removal of a portion of the trophectoderm. In one or more embodiments, piece of the zona pellucida and/or trophectoderm of the first receiver blastocyst may be removed and/or ablated. In one or more embodiments, the breaking of the surface occurs via a laser activation method. In one or more embodiments, the laser activation method includes a first laser, which is a pilot laser, for alignment, and a second laser, which is an ablation laser, for cutting and/or ablation. In one or more embodiments, laser alignment and/or laser ablation is associated with a heating of at least a portion at or near the trophectoderm of the first receiver blastocyst (e.g., proximate the location where the surface of the first receiver blastocyst was broken). In one or more embodiments, a piece of the trophectoderm is ablated. In one or more embodiments, a piece of the trophectoderm is removed via laser activation. This may comprise one laser activation, or may comprise having a first region activated, followed by activation of a second region (blocks 125-128, FIG. 12). In addition, or as an alternative, a piece of the trophectoderm of the first receiver blastocyst may be removed via aspiration and/or suctioning of the zona pellucida and the trophectoderm (block 124, FIG. 12). Preferably, only a small piece of the trophectoderm comprising a few trophectoderm cells are removed and/or ablated (block 129, FIG. 12). In one or more embodiments, only one trophectoderm cell, or only two trophectoderm cells, or, preferably, no more than two, or three, or four trophectoderm cells are removed and/or ablated. In some embodiments, a piece of the trophectoderm comprising only a few or only a small number of trophectoderm cells are removed via a laser activation method (comprising one laser activation, or comprising two laser activations). The laser activation method may or may not include an initial alignment with a pilot laser. In one or more embodiments, one to about 10 cells are removed and/or ablated, or about two cells are removed and/or ablated, or about 3 cells are removed and/or ablated, or from about two to about 6 cells are removed and/or ablated, or from two cells to about five cells are removed and/or ablated, or from one cells to about three cells are removed and/or ablated. In one or more embodiments, the number of trophectoderm cells removed and/or ablated from the first receiver cell is less than the number of trophectoderm cells removed and/or ablated from the first donor cell. In one or more embodiments, the number of trophectoderm cells removed and/or ablated from the first receiver cell is about the same as than the number of trophectoderm cells removed and/or ablated from the first donor cell.

Following or after removal and/or ablation of the first receiver blastocyst in the second manipulation, a piece of the zona pellucida and/or trophectoderm may enter the biopsy needle. The piece of the zona pellucida and/or trophectoderm from the first receiver blastocyst may be expelled and positioned on top of the droplet (block 129, FIG. 12). Ablation and/or loss of the piece of the zona pellucida and/or trophectoderm creates an access from the surface into the first receiver blastocyst. In some embodiments, a supplemental hole may be created in the first receiver blastocyst, which is at or adjacent the location at which the piece of the trophectoderm was removed from the first receiver blastocyst (block 130, FIG. 12). In some embodiments, the supplemental hole is an expansion of the access created by the ablation and/or loss of the piece of the zona pellucida and/or trophectoderm at the surface of the first receiver blastocyst. In some embodiments, when the access is less or smaller than an outer diameter of the biopsy needle, a supplemental hole is created, such as via laser activation and/or laser pulsing, thereby generating the supplemental hole in the outer surface of the first receiver blastocyst and/or ablating a further portion of the outer surface, which generally includes removal and/or ablation of additional portions of the zona pellucida. In one or more embodiments, the supplemental hole is created by utilizing a laser activation method that includes an initial alignment with a first laser, which is a pilot laser, followed by cutting and/or ablation by a second laser, which is an ablation laser. In some embodiments, a hole in at least the zona pellucida of the first receiver blastocyst may be about or similar in size as the outer diameter of the biopsy needle. In one or more embodiments, laser heating associated with laser activation of the first receiver blastocyst may be associated with and/or may improve later attachment and/or proliferation of cells in the first receiver blastocyst (after transfer of donor cells to the first receiver blastocyst). At the completion of the second manipulation of the first receiver blastocyst, the piece of the first receiver blastocyst (the piece comprising the zona pellucida with or without a plurality of trophectoderm cells of the receiver blastocyst) may be discarded.

Referring again to FIG. 11, after or immediately after the first manipulation of the first donor blastocyst, and after or immediately after the second manipulation of the first receiver blastocyst, trophectoderm (comprising the donor cells) mechanically isolated from the first donor blastocyst is provided to the first receiver blastocyst, catalyzing a reaction and/or transforming the first receiver blastocyst (block 102c). Often, transfer of the mechanically isolated trophectoderm (the detached piece comprising the donor trophectoderm cells from the first donor blastocyst), occurs immediately after the second manipulation of the first receiver blastocyst. Transfer comprises introducing the detached piece comprising the donor trophectoderm cells to the first receiver blastocyst. In one or more embodiments, introduction includes holding and/or grasping the first receiver blastocyst with the holding needle (e.g., which may include a mild suctioning for holding of the first receiver blastocyst). In one or more embodiments, introduction of the donor trophectoderm cells includes positioning the biopsy needle and the zona pellucida (at the access hole and/or the supplemental hole) of the first receiver blastocyst in a same visual field. In one or more embodiments, introduction of the donor trophectoderm cells to the first receiver blastocyst includes collecting the detached piece comprising the donor trophectoderm cells in the biopsy needle, ensuring the detached piece comprising the donor trophectoderm cells is near the working end of the biopsy needle (see, e.g., FIG. 14H). In one or more embodiments, introduction of the donor trophectoderm cells to the first receiver blastocyst includes carefully positioning the detached piece comprising the donor trophectoderm cells at the access (with or without the supplemental hole) of the zona pellucida and expelling the detached piece from the biopsy needle so that the detached piece is positioned at or adjacent the access of the first receptor blastocyst (see, e.g., FIGS. 14I, 14J, 14K). In one or more embodiments, introduction of the donor trophectoderm cells to the first receiver blastocyst includes carefully penetrating through the zona pellucida into the first receptor blastocyst. In one or more embodiments, careful penetration may include passing through the hole formed through the zona pellucida. In one or more embodiments, the first receiver blastocyst may be in a collapsed state inside the zona pellucida, or may initially be in a collapsed state inside the zona pellucida. In one or more embodiments, the introduction of the donor trophectoderm cells to the first receiver blastocyst includes utilizing joysticks and suction control to carefully and slowly expel the detached piece comprising the donor trophectoderm cells in a manner that will allow the detached piece comprising the donor trophectoderm cells to locate adjacent trophectoderm cells of the first receiver blastocyst. Introduction of the donor trophectoderm cells to the first receiver blastocyst may, and often, further comprises replacing and/or replenishing at least a portion of the maintenance media of the droplet with new maintenance medium (e.g., comprising at or about 20 wt. % protein, or from about 10 to about 30 wt. % protein). The amount of replacement or replenishment media (new media) will depend on the amount removed. The amount may range from about 2 μl to about 20 μl, or some range or amount therebetween or thereabout. Fresh maintenance media may be introduced by a micropipette and/or needle, or other suitable means that introduces the small amount (e.g., of about 1 μl to about 20 μl, or some range or amount therebetween or thereabout). In some embodiments, such as when there is little loss of maintenance media, an amount of the original maintenance media from the droplet may be removed (e.g., from about 1 μl to about 10 μl, or some range or amount therebetween or thereabout), and subsequently a same, substantially the same, or similar amount of fresh maintenance media (e.g., comprising at or about 20 wt. % protein, or from about 10 to about 30 wt. % protein) may be introduced to the droplet. In one or more embodiments, introduction of the donor trophectoderm cells of the detached piece to the first receiver blastocyst may require some contact of at least one trophectoderm cell of the detached piece with at least one trophectoderm cell of the first receiver blastocyst. In one or more embodiments, prior laser heating of the blastocysts may be associated with and/or may improve later attachment and/or proliferation of cells in the first receiver blastocyst.

Accordingly, as depicted in FIG. 11, blastocyst selection (block 101a), is followed by micromanipulation of the first donor blastocyst (having, for example, one or more of: fair or good trophectoderm cells, and/or a fair or good trophectoderm quality) and micromanipulation of the first receiver blastocyst (having, for example, one or more of: fair or good or less than fair or less than good trophectoderm cells, and/or a fair or good or less than a fair or less than a good trophectoderm quality) (blocks 102a, 102b), with subsequent transfer of trophectoderm cells, and a catalyzing reaction and/or transformation of the blastocyst, including the trophectoderm, in which a transformation occurs, including attachment of trophectoderm cells in the first receiver blastocyst, and proliferation of trophectoderm cells in the first receiver blastocyst (block 102c). In one or more embodiments, the transformation and/or reaction appears sped up for attachment of trophectoderm cells in the first receiver blastocyst, and/or for proliferation of trophectoderm cells in the first receiver blastocyst. It is understood that the laser voltage and pulse widths described herein heat the blastocysts, and cells therein. In one or more embodiments, the transformation and/or reaction may be sped up by the laser heating step(s). In the transformation process and/or reaction that is initiated, the first receiver blastocyst is initiated into generating trophectoderm cells. In one or more embodiments, the transformation process and/or reaction that is initiated includes attachment of newly introduced trophectoderm, from the first donor blastocyst, with trophectoderm of the first receiver blastocyst. In one or more embodiments, the transformation process and/or reaction includes attachment of newly introduced trophectoderm cells, from the first donor blastocyst, with trophectoderm cells of the first receiver blastocyst. In one or more embodiments, the transformation process and/or reaction includes proliferation of the trophectoderm of the first receiver blastocyst. In one or more embodiments, the transformation process and/or reaction is a catalyzing reaction that initiates attachment of trophectoderm cells of the first receiver blastocyst. In one or more embodiments, the transformation process and/or reaction is a catalyzing reaction that initiates and/or promotes proliferation of trophectoderm cells of the first receiver blastocyst. In one or more embodiments, the transformation process and/or reaction provides an increase in viability of the first receiver blastocyst. In one or more embodiments, the transformation process and/or reaction, and/or introducing new trophectoderm cells to the first receiver blastocyst provides to the first receiver blastocyst at least one of the following to the first receiver blastocyst (as or when compared with the first receiver blastocyst before the transformation and/or the reaction, and/or before introducing the new trophectoderm cells to the receiver blastocyst): (i) a high (or higher) number of trophectoderm cells; and/or (ii) a high (or higher) quality of the trophectoderm; (iii) a high (or higher) trophectoderm grade/score; (iv) a high (or higher) ICM grade/rating; and/or (iv) a high (or higher) blastocyst grade/rating.

In one or more embodiments, the systems and methods described herein, and as depicted in FIGS. 10-14, including the transformation process and/or reaction thereof, provide a rescue mechanism for a blastocyst having a low (or lower) blastocyst rating, and/or a low (or lower) trophectoderm morphology, and/or a low (or lower) trophectoderm quality, and/or a low (or lower) trophectoderm grade, and/or a lower (or lower) ICM grade or score. In one or more embodiments, the systems and methods described herein, including the transformation process and/or reaction thereof, provide a cellular composition having undergone the transformation process and/or reaction. In one or more embodiments, the systems and methods described herein, including the transformation process and/or reaction thereof, provide a cellular composition having undergone the transformation process and/or reaction, the cellular composition being in the form of a transformed blastocyst. In one or more embodiments, the transformed blastocyst is a viable blastocyst. In one or more embodiments, the systems and methods described herein, including the transformation process and/or reaction thereof, provide a cellular composition having undergone a catalyzing reaction, the cellular composition being in the form of a catalyzed blastocyst. In one or more embodiments, the blastocyst is a viable blastocyst. In one or more embodiments, the systems and methods described herein, including the transformation process and/or reaction thereof, provide a cellular composition as a viable blastocyst or transformed blastocyst or transformed embryo for transfer. In one or more embodiments, the systems and methods described herein, including the transformation process and/or reaction thereof, provide a cellular composition as a suitable blastocyst with a higher rate of clinical pregnancy and/or birth, as compared with the cellular composition before the transformation process and/or reaction thereof. In one or more embodiments, the systems and methods described herein, including the transformation process and/or reaction thereof, provide a cellular composition or a suitable transformed blastocyst or transformed embryo that is viable for a transfer to a uterus. The transfer may be a single blastocyst transfer, in which the single blastocyst is the cellular composition described herein (e.g., a transformed blastocyst). The transfer may be a double blastocyst transfer, in which at least one of the blastocysts is a cellular composition as described herein (e.g., a transformed blastocyst). The transfer may be a double blastocyst transfer, in which both of the blastocysts are cellular compositions as described herein (e.g., transformed blastocysts). In one or more embodiments, the systems and methods described herein, including the transformation process and/or reaction thereof, provide a cellular composition or suitable transformed blastocyst as described herein that is viable for a fresh transfer to a uterus. In one or more embodiments, the systems and methods described herein, including the transformation process and/or reaction thereof, provide a cellular composition or suitable transformed blastocyst as described herein that is viable for transfer after cryopreservation.

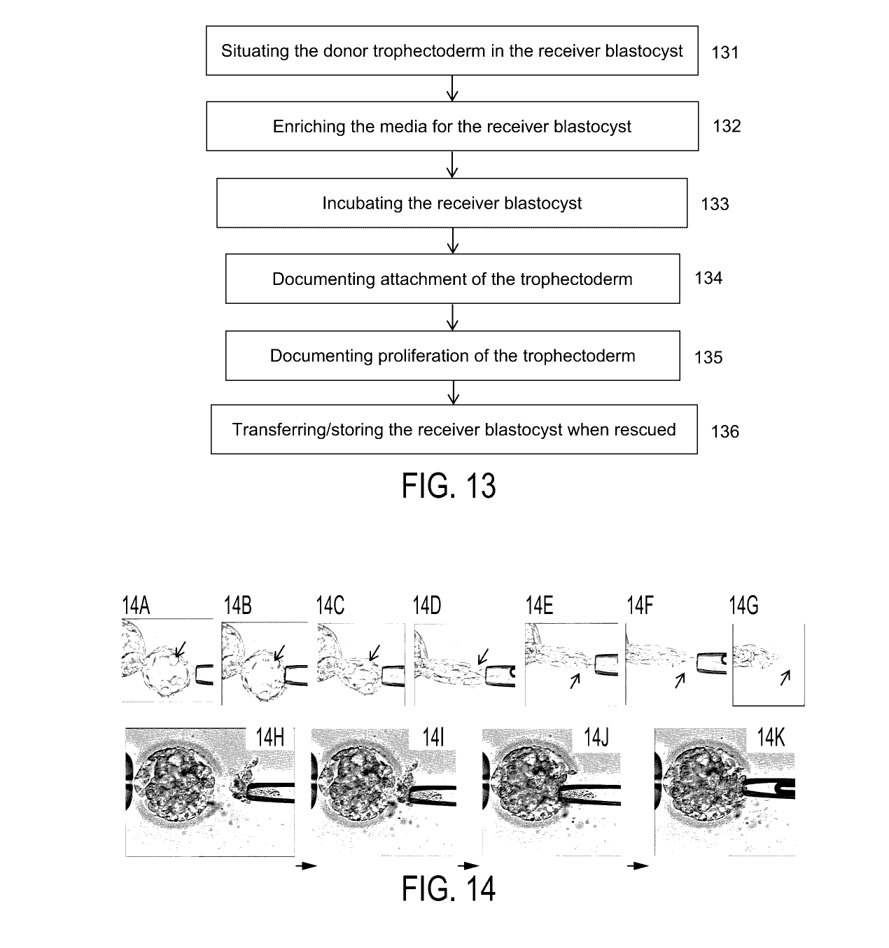

The transformation process and/or reaction thereof for attachment and for proliferation of the trophectoderm cells in the first receiver blastocyst, as generally depicted in FIG. 13, includes at least the microscopic system and the culturing system as described herein. In one or more embodiments, upon or after transfer of the trophectoderm piece from the first donor blastocyst to the first receiver blastocyst (block 102c, FIG. 11), trophectoderm cells from the first donor blastocyst are positioned or repositioned or moved adjacent trophectoderm cells of the first receiver blastocyst, to ensure interaction. In one or more embodiments, after or immediately after transferring the trophectoderm piece from the first donor blastocyst to the first receiver blastocyst (block 131, FIG. 13), at least some fresh maintenance media is provided to the droplet comprising the first receiver blastocyst, to enrich the media and the protein and/or amino acid for the first receiver blastocyst, in which the fresh maintenance media comprises at or about 20 wt. % protein, or from about 10 to about 30 wt. % protein (block 131, FIG. 13). Enrichment with the fresh maintenance media may include adding or replacing from about 1 μl to about 20 μl, or from about 5 μl to about 15 μl, or some range or amount therebetween or thereabout, of the fresh maintenance media. After or immediately after including some fresh maintenance media, the container comprising the droplet and the first receiver blastocyst (comprising the donor trophectoderm cells in the detached piece from the first donor blastocyst) is incubated at a suitable temperature and gas content for living tissue (e.g., at or about 37 degrees Centigrade, or at or about 37.1 degrees Centigrade, with a gas content of about 5% 0₂ and 6% CO₂) (block 133, FIG. 13). The first receiver blastocyst may be monitored periodically thereafter, and images of the first receiver blastocyst may be obtained for analyses (blocks 134, 135). In one or more embodiments, the first receiver blastocyst is monitored every 3 hours for about 9 hours, and thereafter, is monitored at about 20 hours or 24 hours after completion of the donor cell transfer procedure. In one or more embodiments, the first receiver blastocyst is monitored every two to four hours for about 9 to 12 hours, and thereafter, is monitored at about 16 hours, and/or at about 20 hours and/or at about 24 hours after completion of the donor cell transfer procedure. Any variation thereof for monitoring, measurement and/or analyses may be performed to document attachment and to document proliferation of the trophectoderm. Often, proliferation will be observable by about 20 hours, or about 21 hours, or about 22 hours, or about 23 hours, or about 24 hours. Of course, proliferation may be observable earlier than 20 hours. In some embodiments, proliferation may be observable after 24 hours. Pictures and/or video may be utilized to document attachment and document proliferation. In one or more embodiments, an initial measurements of the first receiver blastocyst are obtained and/or recorded, which may include blastocyst area, blastocyst circumference, trophectoderm quality, tight junction quality and/or formation, and/or number of trophectoderm cells. At each monitoring period, similar measurements may be made to document attachment and/or proliferation. In one or more embodiments, an improvement in at least one or more of the measurements is indicative of trophectoderm attachment and proliferation. In one or more embodiments, new growth of the blastocyst and/or new trophectoderm cells is indicative of trophectoderm attachment and/or proliferation. In one or more embodiments, attachment and proliferation is observable at about 14 hours, or about 20 hours, or about 21 hours, or about 22 hours, or about 23 hours, or about 24 hours, or between 14 hours and 26 hours after completion of the donor cell transfer procedure, or any period therebetween or thereabout. Videos and measurements may be recorded, and maintained electronically. The methods described herein may be repeated, for example, utilizing a second donor blastocyst and a second receiver blastocyst, or with as many donor blastocysts and/or receiver blastocysts as needed or desired. Blastocysts may be obtained from any mammal. Each rescued (receiver) blastocyst may be provided a unique identifier. Data elements may be captured that relate to each blastocyst, including but not limited to date of blastocyst collection, number of attempts at attachment, date and time of manipulation and of transfer of the donor cells, blastocyst viability determined at particular time periods (14-26 hours post attachment), and forms of documentation of each blastocyst, including grading or scoring. Rescued blastocysts may be stored after cell attachment. Rescued blastocysts may be utilized or stored after cell proliferation (block 136). In some embodiments, rescued blastocysts that are not utilized at 14 hours, or 20 hours, or 22 hours, or 24 hours, or between 14 and 26 hours after donor cell transfer, may be stored. In some embodiments, rescued blastocysts that are not utilized at 14 hours, or 20 hours, or 22 hours, or 24 hours, or 26 hours after transfer, may be discarded.

Rescued blastocysts that have undergone the mechanical trophectoderm cell transfer, as described herein, have been found to have successful proliferation of trophectoderm cells. In one or more embodiments, such rescued blastocysts may be considered as good quality embryos. The systems and methods described herein may increase the number of blastocysts that may be utilized when retrieved from a subject (mammal). The systems and methods described herein may increase the number of blastocysts that may be utilized in an in vitro fertilization procedure. The systems and methods described herein may increase the number of blastocysts that may be utilized in an artificial insemination procedure. The systems and methods described herein, and the rescued blastocysts produced therefrom, should enhance the number of viable embryos, and should increase considerably the probability of a successful clinical pregnancy as well as a birth outcome.

In a representative example, blastocysts discarded on day 6 and/or after about 140 hours (or between about 120 to 150 hours) post-insemination, and from a single donor dish, were utilized to select both a donor blastocyst and a receptor blastocyst. The selected blastocysts would otherwise have been discarded. After the blastocysts were selected, they were transferred to a study dish that had been labeled for identification purposes only. Data about the donor, the discarded blastocysts, and the selected blastocysts were stored on the computing device and thereafter transferred to a shared drive/server.

The biopsy supplies and equipment included at least the following:
30 degree angled blastomere biopsy needle having a 30 μm ID, flat and polished (#MBB-FP-M-35 from Humagen)
30 degree angled holding needle having 10-30 μm ID (#WHP 120-20 from Wallace Smith Medical)
Stripper tips: 200 μm EZ-Tip from Rocket Medical, Research Instruments Ltd.
Tissue culture dish, 35×10 (#153066 from Nunc, Roskilde, Denmark)
Petri dish, 50 mm×9 mm (#351006 from Falcon)
Ovoil paraffin oil for covering maintenance medium during micromanipulation procedures
Quinn's Advantage™ Medium with HEPES (from Sage In vitro Fertilization, Inc.)
Human Serum Albumin Solution (HSA), 100 mg/ml (from Vitrolife)
G-2™ plus, further supplemented with HSA for culturing blastocysts (e.g., from day 3, including blastocyst stage)
Quinn's Advantage™ Protein Plus (from Sage In vitro Fertilization, Inc.)
Pipette (or Pipette tip holder), 2-20 μl (from Eppendorf)
epT.I.P.S, individually wrapped, 2-200 μl (from Eppendorf)
Inverted microscope with micromanipulators
Saturn Active Laser System

In advance of the blastocyst manipulation, petri dishes were prepared for biopsy by placing two separate droplets (˜10 μl each) of Quinn's Advantage™ Protein Plus. (See also FIG. 9B) The droplets were covered with oil (˜4.5 ml), the lid was placed on the dish, and incubated overnight in a suitable incubator for living tissue. One dish was used for each blastocyst transfer.

One hour prior to performing the biopsy, a supplemental dish was prepared by placing two separate drops (˜10 μl each) of a medium (comprising Quinn's Advantage™ Medium with HEPES also containing 20% protein, such as HSA, and/or G2™ supplemented with HSA having a final concentration of 20% protein), to form a droplet. May also include protein from 10-30 wt. %. This dish was incubated for about 1 hour in a suitable incubator for living tissue, to equilibrate the temperature of the droplet. This dish was utilized for replenishment (replacement) of medium and/or for equilibration of one or more needles.

The two selected blastocyst (receiver blastocyst and donor blastocyst) were positioned in the same droplet. A laser having a power of 400 mW was initiated, in which the laser pulse width was set to 0.606 mS (which had been found to create a hole size of ˜9.5 μm).

The donor blastocyst was manipulated first in accordance with the following steps:

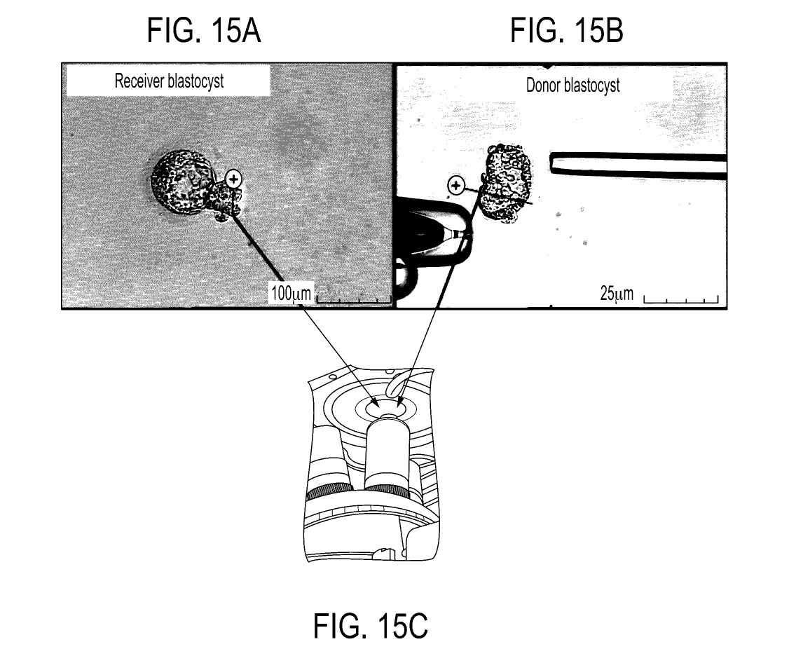

1. The biopsy dish was placed on the micromanipulation microscope stage and the magnification was set to 20×.
2. The holding needle was lowered, and gently grasped the blastocyst to be biopsied (donor blastocyst).
3. The biopsy needle was moved into the same visual field as the blastocyst until it broke the surface of the oil.
4. The biopsy needle was brought into focus using the fine controls and was aligned so as to be only a slight distance from the area of the blastocyst to be biopsied. The biopsy needle and the zona pellucida were within the same visual field.
5. The holding pipette suction control was utilized to gently attach the blastocyst at the site of the ICM.
6. Before attempting to remove the piece of trophectoderm tissue from the blastocyst, the biopsy needle was filled at least partially or fully with the biopsy medium.
7. Using the biopsy pipette, a patch of approximately 5-10 trophectoderm cells was aspirated, so that they cleared the zona pellucida. Suction the patch comprising the trophectoderm cells into the pipette, holding them gently but firmly.
8. Using the two joysticks and suction control, the patch of trophectoderm cells was extended in order to thin the patch.
9. Along a portion of the patch of trophectoderm cells, a laser was aligned at a first region utilizing the red pilot laser, and pulsing of the ablation laser was then initiated. A representative donor blastocyst is shown in FIG. 15B with laser alignment of the red pilot laser, which is also represented in FIG. 15C. When the blastocyst collapsed at the initial pulse of the ablation laser, it re-expanded later.
10. Reapply the ablation laser if a piece of trophectoderm cells at the first region did not detach or separate. More suction pressure may be used to stretch the patch of trophectoderm cells further before the ablation laser was pulsed one more time.
11. All or a piece of the patch of the trophectoderm cells detached, and rapidly entered the biopsy pipet. The detached piece of the trophectoderm cells were expelled from the biopsy needle, and the detached piece was positioned next to the receiver blastocyst in the droplet.

The receiver blastocyst was manipulated in accordance with the following steps:

1. The biopsy dish was placed on the micromanipulation microscope stage, and the magnification was set to 20×.
2. The holding needle was lowered, and gently grasped the blastocyst to be biopsied (receiver blastocyst).
3. The blastocyst was moved into focus.
4. The biopsy needle was lowered into focus, and was aligned, using the fine controls, so that it was a slight distance from the area to be biopsied. The biopsy needle and the zona pellucida were within the same visual field.
5. The blastocyst was gently attached by the holding pipette, utilizing suction control.
6. The biopsy needle was equilibrated with the biopsy medium before attempting to remove any piece of trophectoderm.
7. Use the biopsy needle, 4-5 trophectoderm cells were carefully aspirated, to clear the zona pellucida, and some entered into the pipette, where they were held gently but firmly.
8. The 4-5 trophectoderm cells were extended utilizing two joysticks and suction control in order to thin or stretch the piece formed by the 4-5 trophectoderm cells.
9. The laser was aligned along or on at least one of the 4-5 trophectoderm cells utilizing the red pilot laser, and the ablation laser was then pulsed. A representative receiver blastocyst is shown in FIG. 15A with laser alignment of the red pilot laser, which is also represented in FIG. 15C. Generally, some or all of the blastocyst collapsed at the initial pulse of the ablation laser.
10. If none of the trophectoderm cells detached, or if only a portion detached, more suction pressure was used to further stretch the piece formed by the 4-5 trophectoderm cells, and the ablation laser was pulsed one more time.
11. When the piece formed by the 4-5 trophectoderm cells detached, the piece entered the biopsy pipet, and was quickly expelled on top of the biopsy droplet.
12. After biopsy of the piece formed by the 4-5 trophectoderm cells, the laser was aligned along the zona pellucida utilizing the red pilot laser, near the region where the piece was detached, and the ablation laser was pulsed to make a hole in the zona pellucida, approximately the same size as the biopsy pipette.

The transfer of donor blastocysts cells from the donor blastocyst to the receiver blastocyst was performed as follows:

1. The magnification was set to 20×.
2. The holding needle was lowered, and gently grasped the receiver blastocyst having a low trophectoderm quality.
3. The receiver blastocyst was moved into focus to receive the donor cells.
4. The biopsy needle was lowered into focus using the fine controls, and was aligned at a slight distance from the area to be biopsied. The biopsy needle and the zona pellucida were within the same visual field.
5. The holding needle suction control was used to gently attach and maintain a hold on the blastocyst with the holding needle.
6. The piece of trophectoderm (having the trophectoderm cells) from the donor blastocyst was held by the biopsy needle, and was then move so most of the piece was near the working end of the biopsy needle.
7. The biopsy needle was used to carefully penetrate the zona pellucida of the receiver blastocyst. At this stage the receiver blastocyst was in a collapsed stage inside the zona pellucida.
8. Two joysticks and suction control were used to carefully and slowly expel the piece of trophectoderm (having the trophectoderm cells) so that it was next to the trophectoderm cells of the receiver blastocyst. At least a portion of the trophectoderm cells of the piece of the trophectoderm (of the donor blastocyst) was in contact with trophectoderm cells of the receiver blastocyst when so positioned.

After transfer of the donor cells to the receiver blastocyst, biopsy media in the droplet was immediately replaced using an Eppendorf pipetter (2-20 μl). Approximately, 2-20 μl was replaced by drawing biopsy media out from the droplet, discarding the drawn media, and replenishing the droplet with the same amount of G2™ supplemented with HSA having a final concentration of 20% protein, or from 10-30% protein. The drawn media and the replenishing media should use different Eppendorf tips. Often, about 10 μl of fresh media was removed from the droplet, and was replaced with the fresh protein supplemented media. The dish containing the blastocyst was then placed in the incubator set at 37.1 degrees Centigrade with 5% 0₂ and 6% CO₂. Thereafter, the receiver blastocyst was monitored periodically. Cronus 3 video capture and embryo analysis software was used to obtain video and/or images. Monitoring was every three hours for the first nine hours, and then again at 20 hours post transfer, pictures and videos were taken. Measurement of the blastocyst included area of the blastocyst, outer perimeter of the blastocyst, area of the trophectoderm cells, perimeter of the trophectoderm cells, which were obtained both before and at the periodic intervals after the manipulation and transfer. Further analyses included cell imaging and analyses for cell attachment (e.g., tight junctions), and cell proliferation, and overall cell size and cell number, including new growth. At about 20 or 22 hours after the manipulation and transfer, there was new growth, cell proliferation in the trophectoderm, overall cell growth, and a healthy looking blastocyst, showing success of the procedure.

In one or more embodiments, the systems and methods described herein comprise blastocyst selection, a plurality of blastocyst manipulations, and attachment and proliferation of trophectoderm cells in the selected blastocyst (one originally selected as having low or lower quality trophectoderm, often represented by a low or lower number of trophectoderm cells). Described herein are rescued blastocysts that may be considered viable for a fresh transfer and/or for cryopreservation. The systems and methods described herein provide rescued blastocysts that may be considered viable, thereby having a better rate of clinical pregnancy and/or birth after rescuing. Clinical pregnancy and/or birth outcome may be improved. In one or more embodiments, clinical pregnancy and/or birth outcome may be improved by about or up to about 10%, or by about or up to about 15%, or by about or up to about 20%, or by about or greater than about 15%, as compared with clinical pregnancy and/or birth without the described systems and methods and without the rescued blastocysts described herein.

As used herein and in the appended claims, the singular forms “a,” “and,” and “the” include plural referents unless the context clearly dictates otherwise. Thus, for example, reference to “an agent” or to “a composition” includes a plurality of such agents or compositions, and equivalents thereof known to those skilled in the art, and so forth. It is understood that “comprising” (and related terms such as “comprise” or “comprises” or “having” or “including”) is not intended to exclude embodiments wherein, for example, any composition of matter, composition, method, or process, or the like, described herein may “consist of” or “consist essentially of” the described features. Although representative processes and articles have been described in detail herein, those skilled in the art will recognize that various substitutions and modifications may be made without departing from the scope and spirit of what is described and defined by the appended claims.

What is claimed is:

1. A method of improving quality of a blastocyst, the method comprising:
removing a segment of trophectoderm from a first blastocyst;
removing a portion of trophectoderm from a second blastocyst; and
introducing to the second blastocyst the segment of trophectoderm from the first blastocyst,
the first blastocyst having a same quality or better quality trophectoderm than the second blastocyst.

2. The method of claim 1, wherein the segment of trophectoderm comprises a plurality of trophectoderm cells, at least some of which are united by tight junctions.

3. The method of claim 1, wherein the segment of trophectoderm comprises a plurality of trophectoderm cells united by tight junctions.

4. The method of claim 1, wherein the segment of trophectoderm comprises a plurality of trophectoderm cells, the plurality of trophectoderm cells ranging from about two cells to about twelve cells, or any range therebetween or thereabout.

5. The method of claim 1, wherein the method further comprises breaking a surface of the first blastocyst to remove the segment of trophectoderm from the first blastocyst.

6. The method of claim 1, wherein the method further comprises initially extending the segment of trophectoderm away from a zona pellucida of the first blastocyst before removing the segment of trophectoderm from the first blastocyst.

7. The method of claim 1, wherein the removing the segment of trophectoderm from the first blastocyst comprises:
aligning light emitted from a laser on a first region of the segment of trophectoderm of the first blastocyst,
pulsing light at a frequency and wavelength sufficient to break a portion of the first region;
aligning light emitted from the laser on a second region of the segment of trophectoderm of the first blastocyst; and
pulsing light at a same or similar frequency and wavelength sufficient to break a portion of the second region.

8. The method of claim 1, wherein the removing the portion of trophectoderm from the second blastocyst comprises:
aligning light emitted from a laser on a first region of the portion of trophectoderm of the second blastocyst,
pulsing light at a frequency and wavelength sufficient to break a portion of the first region;
aligning light emitted from the laser on a second region of the portion of trophectoderm of the second blastocyst; and
pulsing light at the same or similar frequency and wavelength sufficient to break a portion of the second region.

9. The method of claim 1, wherein the first blastocyst has a grade of B or C, the grade in accordance with a Gardner grading system or adapted from a Gardner grading system.

10. The method of claim 1, wherein the second blastocyst has a grade of C or D, the grade in accordance with a Gardner grading system or adapted from a Gardner grading system.

11. The method of claim 7, wherein the laser is a high power laser diode emitting light at a wavelength of about 1400 nm, or from about 1000 nm to 2000 nm.

12. The method of claim 8, wherein the laser is a high power laser diode emitting light at a wavelength of about 1400 nm, or from about 1000 nm to 2000 nm.

13. The method of claim 1, wherein the portion of trophectoderm from the second blastocyst includes at or about 3 trophectoderm cells to about 7 trophectoderm cells, or from about 4 to 5 trophectoderm cells.

14. The method of claim 1, wherein the method further comprises placing the first blastocyst and the second blastocyst in a blastocyst media, the blastocyst media further comprising an addition of protein in an amount that is from about 10% to about 30%, or is about 20%.

15. The method of claim 1, wherein the method further comprises placing the second blastocyst in a blastocyst media after introducing to the second blastocyst the segment of trophectoderm from the first blastocyst, the blastocyst media further comprising an addition of protein in an amount that is from about 10% to about 30%, or is about 20%.

16. The method of claim 1, further comprising incubating the second blastocyst for about 14 hours to about 26 hours after the segment of trophectoderm was introduced to the second blastocyst.

17. The method of claim 1, further comprising contacting trophectoderm of the second blastocyst with at least a portion of the segment of trophectoderm when introducing the segment of trophectoderm to the second blastocyst.

18. The method of claim 1, wherein the removing a portion of trophectoderm from the second blastocyst provides access into the second blastocyst, and wherein the access is enlarged by a high power laser ablation.

19. A receiver blastocyst containing trophectoderm cells obtained from a donor blastocyst, the donor blastocyst and the receiver blastocyst from a same fertilization, the donor blastocyst having one or more of a better quality trophectoderm or more trophectoderm cells than the receiver blastocyst before the receiver blastocyst obtained the trophectoderm cells from the donor blastocyst.

20. A blastocyst having an improved quality, the blastocyst being a first blastocyst comprising:
an absence of a portion of a zona pellucida, and an absence of a portion of a trophectoderm, the absence of the portion of the zona pellucida and the absence of the portion of the trophectoderm forming a gap; and
replacement trophectoderm from a second blastocyst located at or adjacent the gap, the replacement trophectoderm comprising trophectoderm of a same or better quality;
the first and second blastocysts being obtained from one or more of a same or similar period of time after fertilization, and a same fertilization.

21. The blastocyst of claim 20, wherein the replacement trophectoderm includes up to ten trophectoderm cells and zona pellucida from the second blastocyst, wherein, in addition to the replacement trophectoderm, the second blastocyst has a higher number of trophectoderm cells than the first blastocyst.

22. A blastocyst system for improving quality of at least one blastocyst, the blastocyst system comprising:
a first blastocyst have a first quality trophectoderm, at least a segment of the first quality trophectoderm removed from the first blastocyst, wherein the segment does not contain cells from an inner cell mass of the first blastocyst; and
the second blastocyst having a second quality trophectoderm, the second quality trophectoderm being one or more of having fewer trophectoderm cells than the first blastocyst, having a same quality trophectoderm as the first blastocyst, and having a lesser quality trophectoderm than the first blastocyst, the second blastocyst further comprising the segment from the first blastocyst in contact with at least a portion of the second quality trophectoderm,
the first and second blastocysts being obtained from one or more of a same or similar period of time after fertilization, and a same fertilization.

23. The blastocyst system of claim 22, further comprising a microscopic system for at least visualizing the first and second blastocysts, the microscopic system comprising micromanipulation tools cooperative with a microinjection system.

24. The blastocyst system of claim 22, further comprising a laser system for at least removing the segment of the first quality trophectoderm from the first blastocyst, and for ablating a portion of the zona pellucida of the second blastocyst.

25. The blastocyst system of claim 22, further comprising a computing system for at least obtaining and storing data about the first and second blastocysts.

26. The blastocyst system of claim 22, further comprising a culturing system for at least culturing the first and second blastocysts, the culturing system including a blastocyst media further comprising an addition of protein in an amount that is from about 10% to about 30%, or is about 20%.
